(19) United States
(12) Patent Application Publication
(10) Pub. No.: US 2010/0056496 A1
(43) Pub. Date: Mar. 4, 2010

(54) MUSCARINIC RECEPTOR ANTAGONISTS

(76) Inventors: Naresh Kumar, Gurgaon (IN); Kirandeep Kaur, Gurgaon (IN); Yogesh D. Shejul, Ahmednagar (IN); Abhijit Ray, New Delhi (IN); Suman Gupta, Gurgaon (IN); Shivani Malhotra, New Delhi (IN); Rajkumar Shirumalla, New Delhi (IN)

Correspondence Address: Ranbaxy Inc., Intellectual Property Department, 600 College Road East, PRINCETON, NJ, 08540, US

(21) Appl. No.: 12/439,771

(22) PCT Filed: Sep. 4, 2007
(86) PCT No.: PCT/IB07/53561

§ 371 Date: Nov. 13, 2009

(30) Foreign Application Priority Data
Sep. 4, 2006 (IN) 1973/DEL/2006

(51) Int. Cl. A61K 31/55; C07D 223/14; A61P 1/00; A61P 11/00; A61P 3/00

(52) U.S. Cl. 514/216; 540/585

(57) ABSTRACT

The present invention generally relates to muscarinic receptor antagonists, which are useful, among other uses, for the treatment of various diseases of the respiratory, urinary and gastrointestinal systems mediated through muscarinic receptors. The present invention also relates to processes for preparing compounds described herein, pharmaceutical compositions thereof, and methods for treating diseases mediated through muscarinic receptors. Formula (I) or a pharmaceutically accepted salt, pharmaceutically acceptable solvate, enantiomers, diastereomer, polymorph or N-oxide thereof, wherein X is.

### FIELD OF THE INVENTION

The present invention generally relates to muscarinic receptor antagonists, which are useful, among other uses, for the treatment of various diseases of the respiratory, urinary and gastrointestinal systems mediated through muscarinic receptors. The present invention also relates to processes for preparing compounds described herein, pharmaceutical compositions thereof, and methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the superfamily of G-protein coupled receptors and five molecularly distinct subtypes are known to exist (M₁, M₂, M₃, M₄ and M₅).

These receptors are widely distributed on multiple organs and tissues and are critical to the maintenance of central and peripheral cholinergic neurotransmission. The regional distribution of these receptor sub-types in the brain and other organs has been documented (for example, the Ml subtype is located primarily in neuronal tissues, such as cerebral cortex and autonomic ganglia, the M₂ subtype is present mainly in the heart and bladder smooth muscle, and the M₃ subtype is located predominantly on smooth muscle and salivary glands (Nature, 323:411 (1986); Science, 237:527 (1987)).

A review in Curr. Opin. Chem. Biol., 3:426 (1999), as well as in Trends in Pharmacol. Sci., 22:409 (2001) by Eglen et al., describes the biological potentials of modulating muscarinic receptor subtypes by ligands in different disease conditions, such as Alzheimer's disease, pain, urinary disease condition, chronic obstructive pulmonary disease, and the like.

The pharmacological and medical aspects of the muscarinic class of acetylcholine agonists and antagonists are presented in a review in Molecules, 6:142 (2001). Birdsall et al. Trends in Pharmacol. Sci., 22:215 (2001) has also summarized the recent developments on the role of different muscarinic receptor subtypes using different muscarinic receptor of knock out mice.

Almost all smooth muscle express a mixed population of M₂ and M₃ receptors. Although M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However the therapeutic utility of antimuscarinics has been limited by poor tolerability as a result of treatment related, frequent systemic adverse events, such as dry mouth, constipation, blurred vision, headache, somnolence and tachycardia. Thus, there exists a need for novel muscarinic receptor antagonists that demonstrate target organ selectivity.

WO 2004/005252 discloses azabicyclo derivatives described as muscarinic receptor antagonists. WO 2004/004629, WO 2004/052857, WO 2004/067510, WO 2004/014853 and WO 2004/014363 disclose 3,6-disubstituted azabicyclo[3.1.0]hexane derivatives described as useful muscarinic receptor antagonists. WO 2004/056811 discloses flaxavate derivatives as muscarinic receptor antagonists. WO 2004/056810 discloses xanthene derivatives as muscarinic receptor antagonists. WO 2004/056767 discloses 1-substituted-3-pyrrolidine derivatives as muscarinic receptor antagonists. WO 99/14200, WO 03/027060, U.S. Pat. No. 6,200,991 and WO 00/56718 disclose heterocycle derivatives as muscarinic receptor antagonists. WO 2004/089363, WO 2004/089898, WO 2004/069835, WO 2004/089900 and WO 2004/089364 disclose substituted azabicyclohexane derivatives as muscarinic receptor antagonists. WO 2006/018708 discloses pyrrolidine derivatives as muscarinic receptor antagonists. WO 2006/035303 discloses azabicyclo derivatives as muscarinic receptor antagonists.

J. Med. Chem., 44:984 (2002), describes cyclohexylmethylpiperidinyl-triphenylpropioamide derivatives as selective M₃ antagonist discriminating against the other receptor subtypes. J. Med. Chem., 36:610 (1993), describes the synthesis and antimuscarinic activity of some 1-cycloalkyl-1-hydroxy-1-phenyl-3-(4-substituted piperazinyl)-2-propanones and related compounds. J. Med. Chem., 34:3065 (1991), describes analogues of oxybutynin, synthesis and antimuscarinic activity of some substituted 7-amino-1-hydroxy-5-heptyn-2-ones and related compounds. Bio-Organic Medicinal Chemistry Letters, 15:2093 (2005) describes synthesis and activity of analogues of oxybutynin and tolterodine. Chem. Pharm. Bull. 53(4):437, 2005 discloses thiazole carboxamide derivatives.

However, there remains a need for novel muscarinic receptor antagonists useful in treating disease states associated with improper smooth muscle function and respiratory disorders.

### SUMMARY OF THE INVENTION

Accordingly, provided herein are novel compounds that can be useful in treating disease states associated with improper smooth muscle function and respiratory disorders.

Thus in one aspect, provided are compounds having the structure of Formula I:

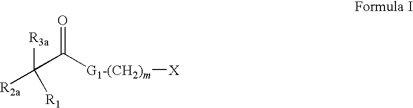

or a pharmaceutically accepted salt, pharmaceutically acceptable solvate, enantiomers, diastereomer, polymorph or N-oxide thereof, wherein
X is

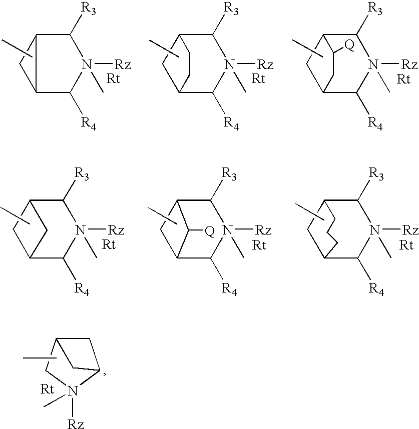

m is an integer selected from 0-4,
Q is alkyl, cycloalkyl, aryl, heteroaryl, heterocyclyl, aralkyl, heteroarylalkyl or heterocyclylalkyl,
G₁ is oxygen, sulfur, —NRᵤ (wherein Rᵤ is hydrogen, alkyl, alkenyl, alkynyl, aryl, or aralkyl) or —CH₂—,
R₁ and R₂ₐ are independently selected from alkyl, alkenyl, alkynyl, cycloalkyl, aryl, aralkyl, heteroarylalkyl or heterocyclylalkyl,
R₃ and R₄ are independently selected from the group consisting of hydrogen, alkyl, alkenyl, alkynyl, cycloalkyl, aryl or aralkyl,
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy,
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring,
Rz is hydrogen or Rq,
Rq is alkyl, alkenyl, alkynyl, cycloalkyl, aryl, heteroaryl, heterocyclyl, heterocyclylalkyl, heteroarylalkyl or aralkyl,
Rt isnoatomor Rq1, and
Rq1 is alkyl, aryl, cycloalkyl, heteroaryl, heterocyclyl, heteroarylalkyl, heterocyclylalkyl or aralkyl,
   wherein when Rz is Rq and Rt is Rq1, then the compound of Formula I is a quaternary ammonium salt.

In another aspect, provided are pharmaceutical compositions comprising a therapeutically effective amount of a compound described herein and one or more pharmaceutically acceptable carriers, excipients or diluents. The pharmaceutical compositions can further comprise one or more corticosteroids, beta agonists, leukotriene antagonists, 5-lipoxygenase inhibitors, anti-histamines, antitussives, dopamine receptor antagonists, chemokine inhibitors, p38 MAP Kinase inhibitors, or PDE-IV inhibitors or a mixture thereof.

In another aspect, provided are methods of treating or preventing disease or disorder of the respiratory, urinary or gastrointestinal systems, wherein the disease or disorder is mediated through muscarinic receptors, comprising administering to a patient in need thereof a therapeutically effective amount of a compound described herein.

In yet another aspect, provided are methods of treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis comprising administering to a patient in need thereof a therapeutically effective amount of a compound described herein.

In another aspect, provided are methods of preparing a compound of Formula V comprising the steps of:

a. reacting a compound of Formula TI

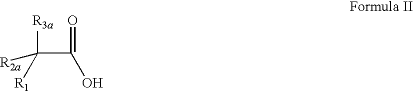

with a compound of Formula III

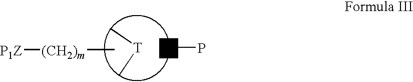

to form a compound of Formula IV, and

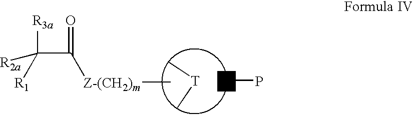

b. deprotecting a compound of Formula IV to form a compound of Formula V,

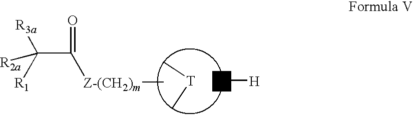

wherein
R₁ and R₂ₐ are independently selected from alkyl, alkenyl, alkynyl, cycloalkyl, aryl, aralkyl, heteroarylalkyl or heterocyclylalkyl;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring;
P₁ is mesyl, tosyl or H when Z is oxygen or —NRᵤ, (wherein Rᵤ, is hydrogen, alkyl, alkenyl, alkynyl, aryl, or aralkyl); or
P₁ is Br, Cl or I when Z is —CH₂;
m is an integer selected from 0-4;
P is a protecting group selected from aralkyl, —C(═O)Oaralkyl, —C(═O)OC(CH₃)₃, —C(═O)OC(CH₃)₂CHBr₂ or —C(═O)OC(CH₃)₂CCl₃;

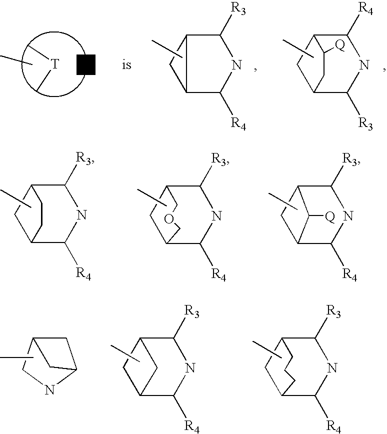

Q is alkyl, cycloalkyl, aryl, heteroaryl, heterocyclyl, aralkyl, heteroarylalkyl or heterocyclylalkyl; and
R₃ and R₄ are independently selected from the group consisting of hydrogen, alkyl, alkenyl, alkynyl, cycloalkyl, aryl or aralkyl.

In another aspect, provided are methods of preparing a compound of Formula VII comprising the step of:

a. reacting a compound of Formula V

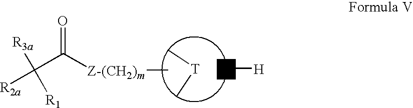

with a compound of Formula VI

Ra-L Formula VI

to form a compound of Formula VII,

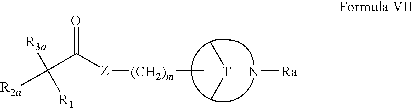

wherein
Z is oxygen or —NRᵤ (wherein Rᵤ is hydrogen, alkyl, alkenyl, alkynyl, aryl or aralkyl) or —CH₂;
R₁ and R₂ₐ are independently selected from alkyl, alkenyl, alkynyl, cycloalkyl, aryl, aralkyl, heteroarylalkyl or heterocyclylalkyl;
Ra is alkyl, alkenyl, alkynyl, heteroarylalkyl, heterocyclylalkyl, aralkyl or cycloalkyl;
L is a leaving group selected from halogen (Cl, Br, I), triflate, tosylate or mesylate;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring;
m is an integer selected from 0-4;

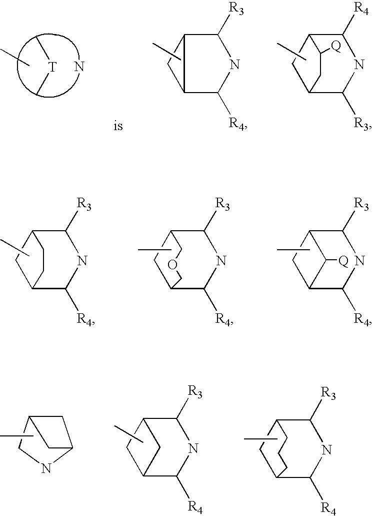

Q is alkyl, cycloalkyl, aryl, heteroaryl, heterocyclyl, aralkyl, heteroarylalkyl or heterocyclylalkyl;
R₃ and R₄ are independently selected from the group consisting of hydrogen, alkyl, alkenyl, alkynyl, cycloalkyl, aryl or aralkyl.

In another aspect, provided are methods of preparing a compound of Formula VIII comprising the step of:

a. reacting a compound of Formula V

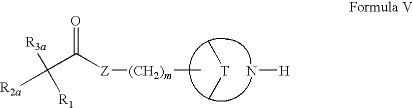

with a compound of Formula Va

Rb-CHO Formula Va

to form a compound of Formula VIII,

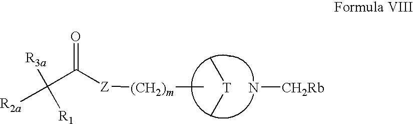

wherein
Z is oxygen or —NRᵤ (wherein Rᵤ is hydrogen, alkyl, alkenyl, alkynyl, aryl, or aralkyl) or —CH₂;
R₁ and R₂ₐ are independently selected from alkyl, alkenyl, alkynyl, cycloalkyl, aryl, aralkyl, heteroarylalkyl or heterocyclylalkyl;
Rb is hydrogen, alkyl, alkenyl, alkynyl, heteroarylalkyl, heterocyclylalkyl, aralkyl or cycloalkyl;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring;
m is an integer selected from 0-4;

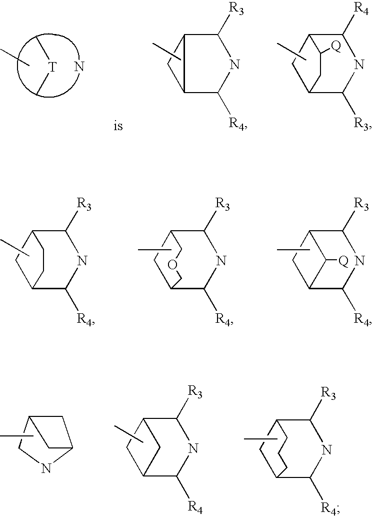

Q is alkyl, cycloalkyl, aryl, heteroaryl, heterocyclyl, aralkyl, heteroarylalkyl or heterocyclylalkyl; and
R₃ and R₄ are independently selected from the group consisting of hydrogen, alkyl, alkenyl, alkynyl, cycloalkyl, aryl or aralkyl.

In yet another aspect, provided are methods of preparing a compound of Formula X comprising the step of:

a. reacting a compound of Formula IX

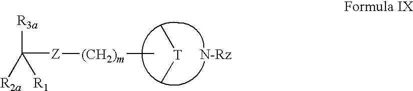

with a compound of Formula Rt-Rc to form a compound of Formula X,

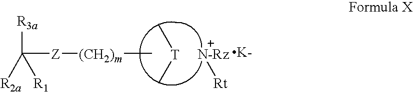

wherein
Z is oxygen or —NRᵤ (wherein Rᵤ is hydrogen, alkyl, alkenyl, alkynyl, aryl, or aralkyl) or —CH₂;
R₁ and R₂ₐ are independently selected from alkyl, alkenyl, alkynyl, cycloalkyl, aryl, aralkyl, heteroarylalkyl or heterocyclylalkyl;
Rt is no atom or Rq1;
Rq1 is alkyl, aryl, cycloalkyl, heteroaryl, heterocyclyl, heteroarylalkyl, heterocyclylalkyl or aralkyl;
Rc is halogen (Cl, Br or I), mesyl, tosyl or triflyl;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring;
Rz is hydrogen or Rq;

Rq is alkyl, alkenyl, alkynyl, cycloalkyl, aryl, heteroaryl, heterocyclyl, heterocyclylalkyl, heteroarylalkyl or aralkyl;
K⁻ is an anion selected from tartrate, chloride, bromide, iodide, sulfate, phosphate, nitrate, carbonate, fumarate, glutamate, citrate, methanesulfonate, benzenesulfonate, maleate or succinate;
m is an integer selected from 0-4;

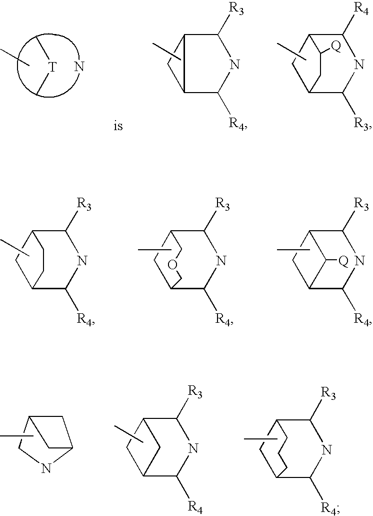

Q is alkyl, cycloalkyl, aryl, heteroaryl, heterocyclyl, aralkyl, heteroarylalkyl or heterocyclylalkyl; and
R₃ and R₄ are independently selected from the group consisting of hydrogen, alkyl, alkenyl, alkynyl, cycloalkyl, aryl or aralkyl.

### DETAILED DESCRIPTION OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with one or more pharmaceutically acceptable carriers, excipients or diluents, which can be useful for the treatment of various diseases of the respiratory, urinary and gastrointestinal systems.

Enantiomers, diastereomers, N-oxides, polymorphs, pharmaceutically acceptable salts and pharmaceutically acceptable solvates of compounds described herein, as well as metabolites having the same type of activity, are also provided, as well as pharmaceutical compositions thereof in combination with one or more pharmaceutically acceptable carriers, excipients or diluents.

Other aspects will be set forth in the description which follows, and in part will be apparent from the description or may be learnt by the practice of the invention.

In accordance with one aspect, there are provided compounds having the structure of Formula I

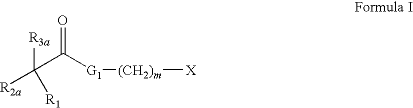

and its pharmaceutically acceptable salts, pharmaceutically acceptable solvates, enantiomers, diastereomers, polymorphs or N-oxides, wherein
X is

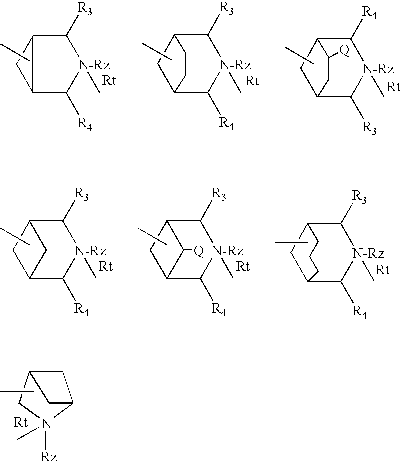

m is an integer selected from 0-4;
Q is alkyl, cycloalkyl, aryl, heteroaryl, heterocyclyl, aralkyl, heteroarylalkyl or heterocyclylalkyl;
G₁ is oxygen, sulfur, —NRᵤ (wherein Rᵤ is hydrogen, alkyl, alkenyl, alkynyl, aryl, aralkyl) or —CH₂—;
R₁ and R₂ₐ are independently selected from alkyl, alkenyl, alkynyl, cycloalkyl, aryl, aralkyl, heteroarylalkyl or heterocyclylalkyl;
R₃ and R₄ are independently selected from the group consisting of hydrogen, alkyl, alkenyl, alkynyl, cycloalkyl, aryl or aralkyl;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring;
Rz is hydrogen or Rq;
Rq is alkyl, alkenyl, alkynyl, cycloalkyl, aryl, heteroaryl, heterocyclyl, heterocyclylalkyl, heteroarylalkyl or aralkyl;

Rt is no atom or Rq1;

Rq1 is alkyl, aryl, cycloalkyl, heteroaryl, heterocyclyl, heteroarylalkyl, heterocyclylalkyl or aralkyl; and wherein when Rz is Rq and Rt is Rq1, then the compound of Formula I is a quaternary ammonium salt.

The following definitions apply to terms as used herein:

The term “alkyl”, unless otherwise specified, refers to a monoradical branched or unbranched saturated hydrocarbon chain having from 1 to 20 carbon atoms. Groups such as methyl, ethyl, n-propyl, iso-propyl, n-butyl, iso-butyl, t-butyl, n-hexyl, n-decyl, tetradecyl, and the like exemplify this term. Alkyl groups may be further substituted with one or more substituents selected from the group consisting of alkenyl, alkynyl, hydroxy, alkoxy, aryloxy, cycloalkyl, acyl, acylamino, acyloxy, —NRₕC(═O)ORⱼ (wherein Rₕ and Rⱼ is the same as defined below), azido, cyano, halogen, thiocarbonyl, substituted thiocarbonyl, carboxy, —COORⱼ (wherein Rⱼ is selected from alkyl, alkenyl, alkynyl, cycloalkyl, aryl, aralkyl, heteroaryl, heterocyclyl, heteroarylalkyl or heterocyclylalkyl), thiol, alkoxyamino, —NRₕRᵢ (wherein Rₕ and Rᵢ are independently selected from hydrogen, alkyl, cycloalkyl, aryl, halogen, aralkyl, heteroaryl, heterocyclyl, heteroarylalkyl or heterocyclylalkyl; Rₕ and Rᵢ may also together join to form a heterocyclyl ring), —C(═O)NRₕRᵢ, —OC(═O)NRₕRᵢ, —NRₕC(═O)NRₕRᵢ, (wherein Rₕ and Rᵢ are the same as defined earlier), nitro, —S(O)ₖRₚ [wherein Rₚ is alkyl, aralkyl, heteroaryl, heterocyclyl, cycloalkyl, heteroaralkyl, heterocyclylalkyl or NRₕRᵢ (wherein Rₕ and Rᵢ are defined earlier) and k is 0, 1 or 2] or —NRₕSO₂Rₚ. Unless otherwise constrained by the definition, all substituents may be further substituted by 1-3 substituents chosen from alkyl, carboxy, —COORⱼ (wherein Rⱼ is the same as defined earlier), —NRₕRᵢ, —C(═O)NRₕRᵢ, —OC(═O)NRₕRᵢ, —NRₕC(═O)NRₕRᵢ, —NRₕC(═O)ORⱼ, (wherein Rⱼ, Rₕ and Rᵢ are the same as defined earlier), hydroxy, alkoxy, halogen, —CF₃, cyano and —S(O)ₖRₚ (where k and Rₚ are the same as defined earlier). Alkyl groups as defined above may also be interrupted by 1-5 atoms of groups independently chosen from oxygen, sulfur and —NRₛ, (where R₁ is alkyl, alkenyl, alkynyl, aryl, cycloalkyl, heteroaryl or heterocyclyl).

The term alkenyl”, unless otherwise specified, refers to a monoradical of a branched or unbranched unsaturated hydrocarbon group preferably having 2 to 20 carbon atoms with cis or trans geometry. Preferred alkenyl groups include ethenyl or vinyl, 1-propylene or allyl, iso-propylene, bicyclo[2.2.1]heptene, and the like. In the event that alkenyl is attached to the heteroatom, the double bond cannot be alpha to the heteroatom. Alkenyl groups may be further substituted with one or more substituents selected from the group consisting of alkyl, alkynyl, alkoxy, acyl, acylamino, acyloxy, —CF₃, —NRₕRᵢ, —C(═O)NRₕRᵢ, —OC(═O)NRₕRᵢ, —NRₕSO₂Rₚ, —NRₕC(═O)NRₕRᵢ (wherein Rₚ, Rₕ and Rᵢ are the same as defined earlier), —NRₕC(═O)ORⱼ, azido, cyano, halogen, hydroxy, thiocarbonyl, substituted thiocarbonyl, carboxy, —COORⱼ (wherein Rⱼ is the same as defined earlier), thiol, aryl, aralkyl, aryloxy, cycloalkyl, heterocyclyl, heteroaryl, heterocyclylalkyl, heteroarylalkyl, alkoxyamino, nitro, S(O)ₖRₚ (wherein k and Rₚ are the same as defined earlier). Unless otherwise constrained by the definition, all substituents may optionally be further substituted by 1-3 substituents chosen from alkyl, carboxy, —COORⱼ (wherein Rⱼ is the same as defined earlier), hydroxy, alkoxy, halogen, —CF₃, cyano, —NRₕRᵢ, —C(═O)NRₕRᵢ, —OC(═O)NRₕRᵢ (wherein Rₕ and Rᵢ are the same as defined earlier) and —S(O)ₖRₚ (where Rₚ and k are the same as defined earlier).

The term “alkynyl”, unless otherwise specified, refers to a monoradical of an unsaturated hydrocarbon, preferably having from 2 to 20 carbon atoms. Preferred alkynyl groups include ethynyl, propargyl or propynyl, and the like. In the event that alkynyl is attached to the heteroatom, the triple bond cannot be alpha to the heteroatom. Alkynyl groups may be further substituted with one or more substituents selected from the group consisting of alkyl, alkenyl, alkoxy, cycloalkyl, acyl, acylamino, alkoxyamino, acyloxy, —NRₕSO₂Rₚ, —NRₕC(═O)ORⱼ, azido, cyano, halogen, hydroxy, thiocarbonyl, substituted thiocarbonyl, —CF₃, carboxy, —COORⱼ (wherein Rⱼ is the same as defined earlier), thiol, aryl, aralkyl, aryloxy, nitro, heterocyclyl, heteroaryl, heterocyclylalkyl, heteroarylalkyl, —NRₕRᵢ, —C(═O)NRₕRᵢ, —OC(═O)NRₕRᵢ, —NRₕC(═O)NRₕRᵢ (wherein Rₕ and Rᵢ are the same as defined earlier), —S(O)ₖRₚ (wherein k and Rₚ are the same as defined earlier). Unless otherwise constrained by the definition, all substituents may optionally be further substituted by 1-3 substituents chosen from alkyl, carboxy, —COORⱼ (wherein Rⱼ is the same as defined earlier), hydroxy, alkoxy, halogen, —CF₃, —NRₕRᵢ, —C(═O)NRₕRᵢ, —OC(═O)NRₕRᵢ (wherein Rₕ and Rᵢ are the same as defined earlier), cyano and —S(O)ₖRₚ (wherein Rₚ and k are the same as defined earlier).

The term “alkoxy”, unless otherwise specified, denotes the group O-alkyl, wherein alkyl is the same as defined above.

The term “aryl”, unless otherwise specified, refers to a carbocyclic aromatic group, for example, phenyl, biphenyl or naphthyl ring and the like optionally substituted with 1 to 3 substituents selected from the group consisting of halogen (F, Cl, Br, I), hydroxy, alkyl, alkenyl, alkynyl, aryl, aralkyl, cycloalkyl, heterocyclyl, heteroaryl, heterocyclylalkyl or heteroarylalkyl, alkoxy, aryloxy, —CF₃, nitro, —NRₕRᵢ, acyl, cyano, acylamino, thiocarbonyl, substituted thiocarbonyl, —C(═O)NRₕRᵢ, —NRₕSO₂Rₚ, —C(═NOH)NH₂, —NRₕC(═O)NRₕRᵢ, —OC(═O)NRₕRᵢ (wherein Rₕ and Rᵢ are the same as defined earlier), carboxy, —S(O)ₖRₚ (wherein Rₚ and k are the same as defined earlier), —COORⱼ (wherein Rⱼ is the same as defined earlier), —NRₕC(═O)ORⱼ. The aryl group may also be fused with a heterocyclic ring, heteroaryl or cycloalkyl ring.

The term “aralkyl”, unless otherwise specified, refers to aryl linked through alkyl (wherein alkyl is the same as defined above) portion and the alkyl portion contains carbon atoms from 1-6 and aryl is as defined above.

The term “cycloalkyl”, unless otherwise specified, refers to cyclic alkyl groups containing 3 to 20 carbon atoms having a single cyclic ring or multiple condensed rings, which may optionally contain one or more olefinic bonds, unless or otherwise constrained by the definition. Such cycloalkyl groups include, by way of example, single ring structures, such as cyclopropyl, cyclobutyl, cyclopentyl, cyclohexyl, cycloheptyl, cyclooctyl, cyclopentenyl, and the like, or multiple ring structures, such as adamantanyl, and bicyclo[2.2.1]heptane, or cyclic alkyl groups to which is fused with an aryl group, for example indane or tetrahydro-naphthalene and the like. Cycloalkyl groups may be further substituted with one or more substituents selected from the group consisting of alkyl, alkenyl, alkynyl, alkoxy, cycloalkyl, acyl, acylamino, acyloxy, aryl aralkyl, —NRₕC(═O)ORⱼ, azido, cyano, halogen, hydroxy, thiocarbonyl, substituted thiocarbonyl, carboxy, —COORⱼ (wherein Rⱼ is the same as defined earlier), thiol, aryl, aralkyl, aryloxy, —NRₕRᵢ, —NRₕC(═O)NRₕRᵢ, —NRₕSO₂Rₚ, —C(═O)NRₕRᵢ, —OC(═O)NRₕRᵢ (wherein Rₕ and Rᵢ are the same as defined earlier), nitro, heterocyclyl, heteroaryl, heterocyclylalkyl, heteroarylalkyl, —CF₃, —S(O)ₖRₚ (wherein Rₚ and k are the same as defined earlier). Unless otherwise constrained by the definition, all substituents may optionally be further substituted by 1-3 substituents chosen from alkyl, carboxy, hydroxy, alkoxy, halogen, CF₃, —NRₕRᵢ, —C(═O)NRₕRᵢ, —NRₕC(═O)NRₕRᵢ, —OC(═O)NRₕRᵢ (wherein Rₕ and Rᵢ are the same as defined earlier), cyano and —S(O)ₖRₚ (wherein Rₚ and k are the same as defined earlier).

The term “carboxy”, unless otherwise specified, as defined herein refers to —C(═O)OH.

The term “aryloxy”, unless otherwise specified, denotes the group O-aryl, wherein aryl is as defined above.

The term “heteroaryl”, unless otherwise specified, refers to monocyclic aromatic ring structure containing 5 or 6 carbon atoms, a bicyclic or a tricyclic aromatic group having 8 to 10 carbon atoms, wherein any one or more carbon atoms of the ring are replaced with one or more heteroatom(s) independently selected from the group consisting of N, O and S. The heteroaryl ring may optionally substituted with 1 to 3 substituent(s) selected from the group consisting of halogen (F, Cl, Br, I), hydroxy, alkoxy, alkyl, alkenyl, alkynyl, cycloalkyl, heterocyclyl, aryl, aralkyl, heteroaryl, heterocyclylalkyl, heteroarylalkyl, acyl, acylamino, thiocarbonyl, substituted thiocarbonyl, alkoxyamino, —NRₕC(═O)ORⱼ, —NRₕSO₂Rₚ, carboxy, —S(O)ₖRₚ (wherein Rₕ, Rⱼ, Rₚ and k are the same as defined earlier), —CF₃, —COORⱼ (wherein Rⱼ is the same as defined earlier), cyano, nitro, —NRₕRᵢ, —C(═O)NRₕRᵢ, —NRₕC(═O)NRₕRᵢ and —OC(═O)NRₕRᵢ (wherein Rₕ and Rᵢ are the same as defined earlier). Examples of heteroaryl groups are pyridinyl, pyridazinyl, pyrimidinyl, pyrrolyl, oxazolyl, thiazolyl, thienyl, isoxazolyl, triazinyl, furanyl, pyrazolyl, imidazolyl, benzimidazolone, pyrazolone, benzofuranyl, indolyl, benzothiazolyl, xanthene, benzoxazolyl, and the like.

The term “heterocyclyl”, unless otherwise specified, refers to a non aromatic monocyclic, bicyclic (fused, bridged, or spiro) or tricyclic cycloalkyl group having 5 to 10 atoms in which 1 to 3 carbon atoms in a ring are replaced by heteroatoms selected from the group comprising of O, S and N, and are optionally benzofused or fused heteroaryl of 5-6 ring members and the heterocyclyl group is optionally substituted, wherein the substituents are selected from the group consisting of halogen (F, Cl, Br, I), hydroxy, alkoxy, alkyl, alkenyl, alkynyl, cycloalkyl, heterocyclyl, aryl, heteroaryl, aralkyl, heterocyclylalkyl, heteroarylalkyl, acyl, acylamino, thiocarbonyl, substituted thiocarbonyl, cyano, alkoxyamino, —NRₕSO₂Rₚ, —NRₕC(═O)ORⱼ, nitro, —CF₃, carboxy, —S(O)ₖRₚ (wherein Rₚ and k are the same as defined earlier), —COORⱼ (wherein Rⱼ is the same as defined earlier), —NRₕC(═O)NRₕRᵢ, —C(═O)NRₕRᵢ, —OC(═O)NRₕRᵢ (wherein Rₕ and Rᵢ are the same as defined earlier). Examples of heterocyclyl groups are tetrahydrofuranyl, dihydrofuranyl, dihydropyridinyl, isoxazolinyl, piperidinyl, morpholine, piperazinyl, dihydrobenzofuryl, azabicyclohexyl, azabicyclooctyl, dihydroindolyl, imidazoline, and the like.

The term “heteroarylalkyl”, unless otherwise specified, refers to heteroaryl (wherein heteroaryl is same as defined earlier) linked through alkyl (wherein alkyl is the same as defined above) portion and the alkyl portion contains carbon atoms from 1-6.

The term “heterocyclylalkyl”, unless otherwise specified, refers to heterocyclyl (wherein heterocyclyl is same as defined earlier) linked through alkyl (wherein alkyl is the same as defined above) portion and the alkyl portion contains carbon atoms from 1-6.

The term “acyl”, unless otherwise specified, refers to —C(═O)R″, wherein R″ is selected from the group hydrogen, alkyl, alkenyl, alkynyl, cycloalkyl, aryl, aralkyl, heteroaryl, heterocyclyl, heteroarylalkyl or heterocyclylalkyl.

The term “thiocarbonyl”, unless otherwise specified, refers to —C(═S)H.

The phrase “substituted thiocarbonyl”, unless otherwise specified, refers to —C(═S)R″, wherein R″ is selected is the same as defined earlier.

The term “leaving group” generally refers to groups that exhibit the desirable properties of being labile under the defined synthetic conditions and also, of being easily separated from synthetic products under defined conditions. Examples of such leaving groups includes, but are not limited to, halogen (F, Cl, Br, I), triflates, tosylate, mesylates, alkoxy, thioalkoxy, hydroxy radicals and the like.

The term “protecting groups” is used herein to refer to known moieties, which have the desirable property of preventing specific chemical reaction at a site on the molecule undergoing chemical modification intended to be left unaffected by the particular chemical modification. Also the term protecting group, unless or other specified may be used with groups, such as hydroxy, amino, carboxy and example of such groups are found in T. W. Greene and P. G. M. Wuts, “Protective Groups in Organic Synthesis”, 2^{nd} Edn. John Wiley and Sons, New York, N.Y., which is incorporated herein by reference. The species of the carboxylic protecting groups, amino protecting groups or hydroxy protecting group employed is not so critical so long as the derivatized moiety/moieties is/are stable to conditions of subsequent reactions and can be removed at the appropriate point without disrupting the remainder of the molecule.

The term “pharmaceutically acceptable salts” refers to derivatives of compounds that can be modified by forming their corresponding acid or base salts. Pharmaceutically acceptable salts may also be formed by complete derivatization of the amine moiety e.g., quaternary ammonium salts.

In accordance with a second aspect, provided are methods for the treatment or prophylaxis of an animal or a human suffering from a disease or disorder of the respiratory, urinary and gastrointestinal systems, wherein the disease or disorder is mediated through muscarinic receptors. The methods include administration of at least one compound having the structure of Formula I.

In accordance with a third aspect, provided are methods for the treatment or prophylaxis of an animal or a human suffering from a disease or disorder associated with muscarinic receptors, comprising administering to a patient in need thereof, an effective amount of a muscarinic receptor antagonist compound of Formula I.

In accordance with a fourth aspect, provided are methods for the treatment or prophylaxis of an animal or a human suffering from a disease or disorder of the respiratory system, such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system, such as irritable bowel syndrome, obesity, diabetes and gastrointestinal hyperkinesis, with one or more compounds of Formula I, wherein the disease or disorder is associated with muscarinic receptors.

In accordance with a fifth aspect, provided are processes for preparing the compounds of Formula I.

The compounds described herein can exhibit significant potency in terms of their activity, as determined by in vitro receptor binding and functional assays and in vivo experiments using anaesthetized rabbits. The compounds that were found active in vitro were tested in vivo. Some of the compounds are potent muscarinic receptor antagonists with high affinity towards M₁ and M₃ receptors than M₂ and/or M₅ receptors. Therefore, pharmaceutical compositions for the treatment for the disease or disorders associated with muscarinic receptors are provided. In addition, the compounds can be administered by any route of administration, including orally or parenterally.

The compounds disclosed herein may be prepared by methods represented by the reaction sequences described herein, for example, as generally shown in Scheme I.

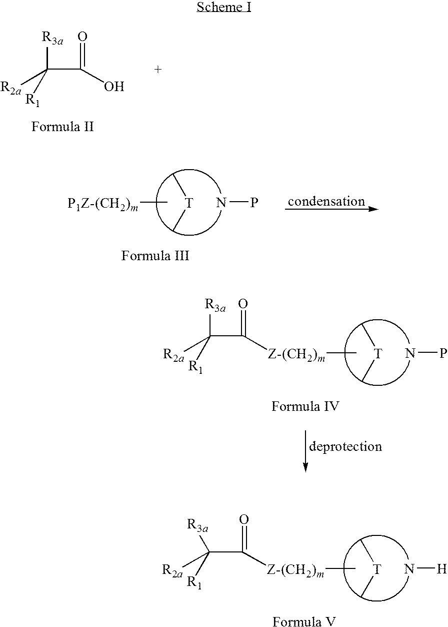

Compounds of Formula V can be prepared following the procedure as described in Scheme I. A compound of Formula II (wherein R₁, R₂ₐ and R₃ₐ are the same as defined earlier) can be reacted with a compound of Formula III [wherein P₁ is mesyl, tosyl or H when Z is oxygen or —NRᵤ (wherein Rᵤ is the same as defined earlier) or P₁ is Br, Cl or I when Z is —CH₂; m is the same as defined earlier; P is a protecting group, for example, aralkyl, —C(═O)Oaralkyl, —C(═O)OC(CH₃)₃, —C(═O)OC(CH₃)₂CHBr₂ or —C(═O)OC(CH₃)₂CCl₃ and

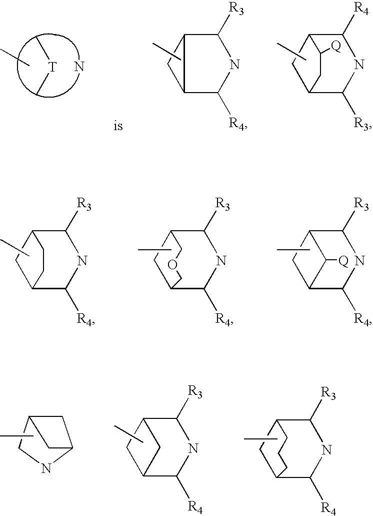

wherein Q is the same as defined earlier] to form a compound of Formula IV. A compound of Formula IV can be deprotected to form a compound of Formula V.

The reaction of a compound of Formula II with a compound of Formula III (when Z is —NRᵤ, wherein Rᵤ is same as defined earlier and PI is H) to form a compound of Formula IV can be carried out in the presence of one or more bases including, for example, N-methylmorpholine, pyridine, triethylamine or diisopropylethylamine. The reaction can also be carried out in the presence of one or more condensing agents, for example, 1-(3-dimethylaminopropyl)-3-ethyl carbodiimide hydrochloride or dicyclohexylcarbodiimide. The reaction can be carried out in one or more organic solvents, for example, dimethylformamide, tetrahydrofuran, dioxane or chloroform.

The reaction of a compound of Formula II with a compound of Formula III (when Z is oxygen and PI is halogen, mesyl or tosyl) to form a compound of Formula IV can be carried out in the presence of one or more bases, for example, 1,8-diazabicyclo[5.4.0]undec-7-ene, N-methylmorpholine, triethylamine or diisopropylethylamine. This reaction can also be carried out in one or more organic solvents, for example, toluene, benzene or xylene.

Alternatively, Formula II can be reacted with a compound of Formula III (when Z is oxygen and P₁ is H) in the presence of one or more carbonylating agents including, for example, carbonyldiimidazole. This reaction can be carried out in one or more organic solvents, for example, dimethylformamide, tetrahydrofuran or chloroform.

The deprotection of a compound of Formula IV (wherein P is aralkyl) to form a compound of Formula V can be carried out in the presence of one or more deprotecting agents (for example, palladium on carbon in presence of hydrogen gas or a hydrogen source, such as ammonium formate solution, formic acid or cyclohexene). The deprotection reaction can be carried out in one or more organic solvents (for example, methanol, ethanol, propanol or isopropylalcohol).

Alternatively, deprotection of a compound of Formula IV (wherein P is aralkyl) to form a compound of Formula V can be carried out in the presence of one or more chloroformates. The deprotection can also be carried out in one or more organic solvents (for example, dichloromethane, dichloroethane, carbon tetrachloride or chloroform).

The deprotection of a compound of Formula IV (wherein P is —C(═O)Oaralkyl) to form a compound of Formula V can be carried out in an alkaline alcoholic solution containing one or more bases. Suitable bases include, for example, potassium hydroxide, sodium hydroxide or lithium hydroxide. Suitable alcohols include, for example, methanol, ethanol, propanol, diethylether or isopropylalcohol.

The deprotection of a compound of Formula IV (when P is —C(═O)Oaralkyl) can be carried out in the presence of one or more deprotecting agents (for example, palladium on carbon in presence of hydrogen gas or palladium on carbon with a source of hydrogen gas (for example, ammonium formate solution, cyclohexene or formic acid)). The deprotection can also be carried out in one or more organic solvents (for example, methanol, ethanol, propanol or isopropylalcohol).

The deprotection of a compound of Formula IV (wherein P is —C(═O)OC(CH₃)₃ or —C(═O)OC(CH₃)₂CHBr₂) to form a compound of Formula V can be carried out in an alcoholic solution containing one or more acids or trifluoroacetic acid in dichloromethane. Suitable alcohols include, for example, hydrochloric acid solution of methanol, ethanol, propanol, isopropylalcohol, ethylacetate or ether.

The deprotection of a compound of Formula IV (wherein P is —C(═O)OC(CH₃)₂CCl₃) to form a compound of Formula V can be carried out in the presence of one or more supernucleophiles (for example, lithium cobalt (I) phthalocyanine, zinc and acetic acid or cobalt phthalocyanine).

Examples of compounds include:
(3-Benzyl-3-azabicyclo[3.2.1]oct-8-yl)methyl cyclohexyl(hydroxy)phenylacetate (Compound No. 11),
(2R)—N-[(3-Benzyl-3-azabicyclo[3.2.1]oct-8-yl)methyl]-2-cyclopentyl-2-hydroxy-N-methyl-2-phenylacetamide (Compound No 14),
(3-Benzyl-3-azabicyclo[3.2.1]oct-8-yl)methyl hydroxy(4-methylphenyl)phenylacetate (Compound No. 16),
3-Hydroxy-N-{[3-(4-methylbenzyl)-3-azabicyclo[3.2.1]oct-8-yl]methyl}-2-phenylpropanamide (Compound No. 24),
(3-Benzyl-3-azabicyclo[3.2.1]oct-8-yl)methyl cycloheptyl(hydroxy)-2-thienylacetate (Compound No. 25),
(3-Benzyl-3-azabicyclo[3.2.1]oct-8-yl)methyl cyclohexyl(hydroxy)-2-thienylacetate (Compound No. 26),
(3-Benzyl-3-azabicyclo[3.2.1]oct-8-yl)methyl cycloheptyl(hydroxy)phenylacetate (Compound No. 27),
(3-Benzyl-3-azabicyclo[3.2.1]oct-8-yl)methyl (4-fluorophenyl)(hydroxy)phenylacetate (Compound No. 28),
(3-Benzyl-3-azabicyclo[3.2.1]oct-8-yl)methyl (2R)-[(1R)-3,3-difluorocyclohexyl](hydroxy)phenylacetate (Compound No. 43),
   and pharmaceutically accepted salts, pharmaceutically acceptable solvates, enantiomers, diastereomers, polymorphs or N-oxides thereof

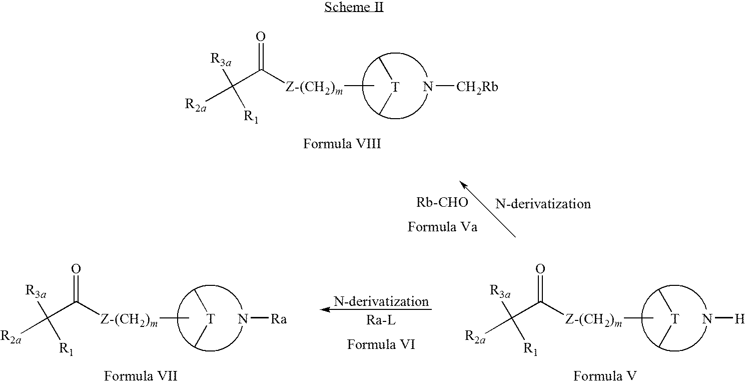

Compounds of Formulae VII and VIII can be prepared following the procedure as depicted in Scheme II.

A compound of Formula V (wherein

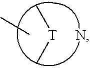

Z, R₁, R₂ₐ, R^{3a}, Rz, m and T are the same as defined earlier) can be reacted with a compound of Formula VI (wherein Ra is alkyl, alkenyl, alkynyl, heteroarylalkyl, heterocyclylalkyl, aralkyl or cycloalkyl and L is the leaving group which is same as defined earlier) to form a compound of Formula VII. The reaction can be carried out in the presence of one or more bases. Suitable bases include, for example, potassium carbonate, sodium carbonate or sodium bicarbonate. The reaction can also be carried out in one or more organic solvents. Suitable organic solvents include, for example, dimethylformamide, acetone, acetonitrile, dichloromethane, chloroform or carbon tetrachloride.

A compound of Formula V (wherein R₁, R₂ₐ, R₃ₐ, Rz, m and T are the same as defined earlier) can be reacted (by reductive amination) with a compound of Formula Va (wherein Rb is hydrogen, alkyl, alkenyl, alkynyl, heteroarylalkyl, heterocyclylalkyl, aralkyl or cycloalkyl) to form a compound of Formula VII. The reaction can be carried out in the presence of one or more reducing agents. Suitable reducing agents include, for example, sodium cyanoborohydride or sodium triacetoxyborohydride. The reaction can also be carried out in one or more organic solvents. Suitable solvents include, for example, acetonitrile or dichloromethane or tetrahydrofuran.

Examples of compounds include:
(3-Methyl-3-azabicyclo[3.2.1]oct-8-yl)methyl hydroxy(4-methylphenyl)phenylacetate (Compound No. 29),
(3-Methyl-3-azabicyclo[3.2.1]oct-8-yl)methyl cycloheptyl(hydroxy)phenylacetate (Compound No. 30),
(3-Methyl-3-azabicyclo[3.2.1]oct-8-yl)methyl (4-fluorophenyl)(hydroxy)phenylacetate (Compound No. 31),
(3-Methyl-3-azabicyclo[3.2.1]oct-8-yl)methyl (2R)-[(1R)-3,3-difluorocyclopentyl](hydroxy)phenylacetate (Compound No. 32),
(3-Methyl-3-azabicyclo[3.2.1]oct-8-yl)methyl cyclohexyl(hydroxy)phenylacetate (Compound No. 33),
(3-Methyl-3-azabicyclo[3.2.1]oct-8-yl)methyl (2R)-[(1S)-3,3-difluorocyclopentyl](hydroxy)phenylacetate (Compound No. 44),
(3-Methyl-3-azabicyclo[3.2.1]oct-8-yl)methyl (2R)-[(1R)-3,3-difluorocyclohexyl](hydroxy)phenylacetate (Compound No. 45),
(3-Methyl-3-azabicyclo[3.1.0]hex-6-yl)methyl (2R)-[(1S)-3,3-difluorocyclohexyl](hydroxy)phenylacetate (Compound No. 46),
(3-Methyl-3-azabicyclo[3.1.0]hex-6-yl)methyl (2R)-(3,3-difluorocyclopentyl)(hydroxy)phenylacetate (Compound No. 47),
   and pharmaceutically accepted salts, pharmaceutically acceptable solvates, enantiomers, diastereomers, polymorphs or N-oxides thereof.

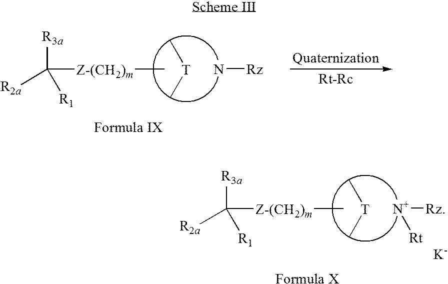

Compounds of Formula X can be prepared following the procedure as described in Scheme III.

A compound of Formula IX (wherein Z, m, Rz, R₁, R₂ₐ and R₃ₐ are the same as defined earlier) can be reacted with a compound of Formula Rt-Rc (wherein Rt is the same as defined earlier and Rc is halogen (Cl, Br or I), mesyl, tosyl or triflyl) to form a compound of Formula X (wherein K⁻ is an anion disclosed in International Journal of Pharmaceutics, 33 (1986), page 202, for example, but not limited to, tartrate, chloride, bromide, iodide, sulfate, phosphate, nitrate, carbonate, fumarate, glutamate, citrate, methanesulfonate, benzenesulfonate, maleate or succinate). The reaction can be carried out in one or more organic solvents, for example, dichloromethane, dichloroethane, carbon tetrachloride, chloroform, methanol, dimethylformamide or acetonitrile.

Examples of compounds include:
Iodide salt of 3-benzyl-6-({[cyclopentyl(hydroxy)-2-thienylacetyl]amino}methyl)-3-methyl-3-azoniabicyclo[3.1.0]hexane (Compound No. 1),
Iodide salt of 3-cyclohexylmethyl-6-({[cyclopentyl(hydroxy)phenylacetyl]amino}methyl)-3-methyl-3-azoniabicyclo[3.1.0]hexane (Compound No. 2),
Iodide salt of 6-({[hydroxy(3-methylphenyl)phenylacetyl]oxy}methyl)-3,3-dimethyl-3-azoniabicyclo[3.1.0]hexane (Compound No. 3),
Iodide salt of 3-benzyl-6-({[cyclopentyl(hydroxy)(4-methylphenyl)acetyl]amino}methyl)-3-methyl-3-azoniabicyclo[3.1.0]hexane (Compound No. 4),
Iodide salt of 6-({[hydroxy(diphenyl)acetyl]amino}methyl)-3-methyl-3-(4-methylbenzyl)-3-azoniabicyclo[3.1.0]hexane (Compound No. 5),
Iodide salt of 6-({[cyclopentyl(hydroxy)phenylacetyl]amino}methyl)-3-methyl-3-(3-methylbenzyl)-3-azoniabicyclo[3.1.0]hexane (Compound No. 6),
Iodide salt of 3-Benzyl-6-({[hydroxy(4-methylphenyl)phenylacetyl]oxy}methyl)-3-methyl-3-azoniabicyclo[3.1.0]hexane (Compound No. 7),
Iodide salt of 3-benzyl-6-({[cyclopentyl(hydroxy)-2-thienylacetyl]oxy}methyl)-3-methyl-3-azoniabicyclo[3.1.0]hexane (Compound No. 8),
Iodide salt of 3-benzyl-6-({[hydroxy(2-dithienyl)acetyl]amino}methyl)-3-methyl-3-azoniabicyclo[3.1.0]hexane (Compound No. 9),
Iodide salt of 3-benzyl-8-({[cyclohexyl(hydroxy)phenylacetyl]oxy}methyl)-3-methyl-3-azoniabicyclo[3.2.1]octane (Compound No. 10),
Bromide salt of 3-benzyl-6-({[hydroxy(phenyl)-2-thienylacetyl]oxy}methyl)-3-methyl-3-azoniabicyclo[3.1.0]hexane (Compound No. 12),
Iodide salt of 3-(3-fluorobenzyl)-6-({[hydroxy(diphenyl)acetyl]amino}methyl)-3-methyl-3-azoniabicyclo[3.1.0]hexane (Compound No. 13),
Iodide salt of 3-benzyl-8-{[[(2R)-2-cyclopentyl-2-hydroxy-2-phenylacetyl](methyl)amino]methyl}-3-methyl-3-azoniabicyclo[3.2.1]octane (Compound No. 15),
Iodide salt of 3-benzyl-8-({[hydroxy(4-methylphenyl)phenylacetyl]oxy}methyl)-3-methyl-3-azoniabicyclo[3.2.1]octane (Compound No. 17),
Iodide salt of 3-benzyl-6-({[(4-fluorophenyl)(hydroxy)phenylacetyl]amino}methyl)-3-methyl-3-azoniabicyclo[3.1.0]hexane (Compound No. 18),
Iodide salt of 3-benzyl-6-({[(4-fluorophenyl)(hydroxy)phenylacetyl]oxy}methyl)-3-methyl-3-azoniabicyclo[3.1.0]hexane (Compound No. 19),
Iodide salt of 6-({[cyclopentyl(hydroxy)phenylacetyl]amino}methyl)-3-methyl-3-(4-methylbenzyl)-3-azoniabicyclo[3.1.0]hexane (Compound No. 20),
Iodide salt of 6-[({(2R)-2-[(1S)-3,3-difluorocyclohexyl]-2-hydroxy-2-phenylacetyl}oxy)methyl]-3,3-dimethyl-3-azoniabicyclo[3.10]hexane (Compound No. 21),
Iodide salt of 6-({[(2R)-2-(3,3-difluorocyclopentyl)-2-hydroxy-2-phenylacetyl]oxy}methyl)-3,3-dimethyl-3-azoniabicyclo[3.10]hexane (Compound No. 22),
Iodide salt of 8-{[(3-hydroxy-2-phenylpropanoyl)amino]methyl}-3-methyl-3-(4-methylbenzyl)-3-azoniabicyclo[3.2.1]octane (Compound No. 23),
Iodide salt of 3-Benzyl-8-({[2-cycloheptyl-2-hydroxy-2-(2-thienyl)acetyl]oxy}methyl)-3-methyl-3-azoniabicyclo[3.2.1]octane (Compound No. 34),
Iodide salt of 3-benzyl-8-({[2-cyclohexyl-2-hydroxy-2-(2-thienyl)acetyl]oxy}methyl)-3-methyl-3-azoniabicyclo[3.2.1]octane (Compound No. 35),
Iodide salt of 3-benzyl-8-({[2-cycloheptyl-2-hydroxy-2-phenylacetyl]oxy}methyl)-3-methyl-3-azoniabicyclo[3.2.1]octane (Compound No. 36),
Iodide salt of 3-benzyl-8-({[(4-fluorophenyl)(hydroxy)phenylacetyl]oxy}methyl)-3-methyl-3-azoniabicyclo[3.2.1]octane (Compound No. 37),
Iodide salt of 8-({[hydroxy(4-methylphenyl)phenylacetyl]oxy}methyl)-3,3-dimethyl-3-azoniabicyclo[3.2.1]octane (Compound No. 38),
Iodide salt of 8-({[cycloheptyl(hydroxy)phenylacetyl]oxy}methyl)-3,3-dimethyl-3-azoniabicyclo[3.2.1]octane (Compound No. 39),
Iodide salt of 8-({[(4-fluorophenyl)(hydroxy)phenylacetyl]oxy}methyl)-3,3-dimethyl-3-azoniabicyclo[3.2.1]octane (Compound No. 40),
Iodide salt of 8-[({(2R)-2-[(1R)-3,3-difluorocyclopentyl]-2-hydroxy-2-phenylacetyl}oxy)methyl]-3,3-dimethyl-3-azoniabicyclo[3.2.1]octane (Compound No. 41)
Iodide salt of 8-({[cyclohexyl(hydroxy)phenylacetyl]oxy}methyl)-3,3-dimethyl-3-azoniabicyclo[3.2.1]octane (Compound No. 42),
Iodide salt of 8-[({(2R)-2-[(1S)-3,3-difluorocyclopentyl]-2-hydroxy-2-phenylacetyl}oxy)methyl]-3,3-dimethyl-3-azoniabicyclo[3.2.1]octane (Compound No. 48),
Iodide salt of 8-[({(2R)-2-[(1R)-3,3-difluorocyclohexyl]-2-hydroxy-2-phenylacetyl}oxy)methyl]-3,3-dimethyl-3-azoniabicyclo[3.2.1]octane (Compound No. 49),
Iodide salt of 3-benzyl-8-[({(2R)-2-[(1R)-3,3-difluorocyclohexyl]-2-hydroxy-2-phenylacetyl}oxy)methyl]-3-methyl-3-azoniabicyclo[3.2.1]octane (Compound No. 50),
   and pharmaceutically accepted salts, pharmaceutically acceptable solvates, enantiomers, diastereomers, polymorphs or N-oxides thereof.

In the above schemes, where specific reactants, bases, condensing agents, protecting groups, deprotecting agents, solvents, catalysts, temperatures, etc. are disclosed, it is to be understood that other bases, condensing agents, protecting groups, deprotecting agents, solvents, catalysts, temperatures, etc. known to those skilled in the art may be used. Similarly, reaction conditions, such as temperature and duration, may be adjusted accordingly.

Suitable salts of the compounds represented by the Formula I were prepared to solubilize such compounds in aqueous medium (for example, for biological evaluations), to be compatible with various dosage formulations and/or aid in the bioavailability of such compounds. Such salts include pharmacologically acceptable salts, such as inorganic acid salts (for example, hydrochloride, hydrobromide, sulfate, nitrate and phosphate) and organic acid salts (for example, acetate, tartrate, citrate, fumarate, maleate, toluenesulfonate and methanesulfonate). When carboxyl groups are present as substituents in the compounds described herein, they may be in the form of an alkaline or alkali metal salt (for example, sodium, potassium, calcium, magnesium, and the like). These salts may be prepared by various techniques, such as treating a compound with an equivalent amount of inorganic or organic acid or base in a suitable solvent.

The compounds described herein include their enantiomers, diastereomers, N-oxides, polymorphs, solvates and pharmaceutically acceptable salts, as well as metabolites having similar type of activity. Pharmaceutical compositions comprising compounds of Formula I or metabolites, enantiomers, diastereomers, N-oxides, polymorphs, solvates or pharmaceutically acceptable salts thereof in combination with one or more pharmaceutically acceptable carriers, excipients or diluents are also provided.

Where desired, compounds of Formula I and/or their pharmaceutically acceptable salts, pharmaceutically acceptable solvates, stereoisomers, tautomers, racemates, prodrugs, metabolites, polymorphs or N-oxides may be advantageously used in combination with one or more other therapeutic agents. Examples of other therapeutic agents include, but are not limited to, corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

The compositions can be administered by route of administration, including, for example, inhalation, insufflation, orally, rectally, parenterally (intravenously, intramuscularly or subcutaneously), intracisternally, intravaginally, intraperitoneally or topically.

Compositions for inhalation or insufflation include solutions and suspensions in pharmaceutically acceptable, aqueous or organic solvents, or mixtures thereof, and powders. Liquid or solid compositions may contain suitable pharmaceutically acceptable excipients. The compositions can be administered by the nasal respiratory route for local or systemic effect. Compositions can be nebulized by use of inert gases. Nebulized solutions may be inhaled directly from a nebulizing device or the nebulizing device can be attached to a face mask tent, or an intermittent positive pressure breathing machine. Solution, suspension, or powder compositions can be administered nasally from devices.

Solid dosage forms for oral administration may be presented in discrete units, for example, capsules, cachets, lozenges, tablets, pills, powders, dragees or granules, each containing a predetermined amount of one or more active compounds (i.e., at least a compound described herein). In such solid dosage forms, the active compound can be admixed with one or more inert excipient (or carrier or diluent), such as sodium citrate or dicalcium phosphate or (a) fillers or extenders, for example, starches, lactose, sucrose, glucose, mannitol and silicic acid, (b) binders, for example, carboxymethylcellulose, alginates, gelatin, polyvinylpyrrolidone, sucrose and acacia, (c) humectants, for example, glycerol, (d) disintegrating agents, for example, agar-agar, calcium carbonate, potato or tapioca starch, alginic acid, certain complex silicates and sodium carbonate, (e) solution retarders, for example paraffin, (f) absorption accelerators, for example, quaternary ammonium compounds, (g) wetting agents, for example, cetyl alcohol and glycerol monostearate, (h) adsorbents, for example, kaolin and bentonite, and (i) lubricants, for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type also include soft and hard-filled gelatin capsules using excipients, for example lactose or milk sugar, as well as high molecular weight polyethylene glycols, and the like.

Solid dosage forms can be prepared with one or more coatings or shells, such as enteric coatings and others well-known in this art. Solid dosage forms may contain opacifying agents, and formulated to release one or more active compounds in a specific part of the gastrointestinal tract, i.e., in a controlled delayed manner. Examples of embedding compositions, which can be used, include polymeric substances and waxes.

Active compounds can also be in micro-encapsulated form, if appropriate, with one or more carriers, excipients or diluents.

Liquid dosage forms for oral administration include pharmaceutically acceptable emulsions, solutions, suspensions, syrups and elixirs. Liquid dosage forms contain may one or more inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan and the like or mixtures thereof.

Such compositions described herein can also include one or more adjuvants, for example, wetting agents, emulsifying and suspending agents, sweetening, flavoring and perfuming agents, colorants or dyes.

Suspensions may contain one or more suspending agents, for example, ethoxylated isostearyl alcohols, polyoxyethylene sorbitol and sorbitan esters, microcrystalline cellulose, aluminum metahydroxide, bentonite, agar-agar and tragacanth, and the like or mixtures thereof.

Dosage forms for topical administration include powders, sprays, inhalants, ointments, creams, salves, jellies, lotions, pastes, gels, aerosols, or oils. Active components can be admixed under sterile conditions with one or more pharmaceutically acceptable carriers, excipients or diluents and optionally one or more preservatives, buffers or propellants. Ophthalmic formulations, eye ointments, powders and solutions are also encompassed herein.

Compositions suitable for parenteral injection may comprise pharmaceutically acceptable sterile aqueous or nonaqueous solutions, dispersions, suspensions or emulsions and sterile powders for reconstitution into sterile injectable solutions or dispersions. These preparations may contain anti-oxidants, buffers, bacteriostats and solutes, which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters, such as ethyl oleate. Proper fluidity can be maintained, for example, by the use of a coating (e.g., lecithin), by the maintenance of the required particle size in the case of dispersions and by the use of surfactants.

Such compositions may also contain adjuvants, such as preserving, wetting, emulsifying, and dispensing agents. Various antibacterial and antifungal agents, for example, parabens, chlorobutanol, phenol, sorbic acid, and the like, may be used, in particular to prevent microorganisms activity. Such compositions may also include isotonic agents, for example sugars, sodium chloride and the like. Prolonged absorption of injectable compositions can be facilitated by the use of agents delaying absorption, for example, aluminum monostearate and gelatin.

Suppositories for rectal administration can be prepared by mixing active ingredients with one or more suitable nonirritating excipients, such as cocoa butter and polyethylene glycols or a suppository wax, which are solid at ambient temperatures but liquid at body temperatures and thus melts in the rectum or vaginal cavity to release the active ingredients.

Compounds described herein can be incorporated into slow-release or targeted delivery systems, such as polymer matrices, liposomes, and microspheres. The compounds may be sterilized, for example, by filtration through a bacteria-retaining filter, or by incorporating sterilizing agents in the form of sterile solid compositions, which can be dissolved in sterile water, or some other sterile injectable medium immediately before use.

Actual dosage levels of active ingredient in the compositions described herein and administration schedules of individual dosages may be readily varied to provide an effective amount of active ingredient that facilitates a desired therapeutic response for a particular composition and route of administration. It is to be understood, however, that specific dosage levels for any particular patient can depend upon a variety of factors including, for example, the body weight, general health, sex and diet of the patient; specific compound chosen; route of administration; the desired duration of treatment; rates of absorption and excretion; combination with other drugs and the severity of the particular disease being treated and is ultimately at the discretion of the physician.

Pharmaceutical compositions described herein can be produced and administered in dosage units, each unit containing a therapeutically effective amount of one or more compound described herein and/or at least one pharmaceutically acceptable addition salt thereof. The dosage may be varied over wide limits as the compounds can be effective at low dosage levels and relatively free of toxicity. Such compounds may be administered in low micromolar concentrations, which amounts are therapeutically effective, and the dosage may be increased accordingly up to the maximum dosage tolerated by the patient.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Scheme I

### Example 1

### Synthesis of (3-benzyl-3-azabicyclo[3.2.1]oct-8-yl)methylcyclohexyl(hydroxy)phenylacetate (Compound No. 11)

1,8-diazabicyclo[5.4.0]undec-7-ene (0.237 g, 1.56 mmol) was added to a solution of 2-hydroxy-2-cyclohexyl phenyl acetic acid (0.27 g, 1.17 mmol) and 3-benzyl-8-[(methylsulfonyl)methyl]-3-azabicyclo[3.2.1]octane (0.3 g, 0.78 mmol) intoluene (15 mL) and the reaction mixture was refluxed for 10 hours. The reaction mixture was concentrated under reduced pressure and a residue thus obtained was purified by preparative thin layer chromatography to yield the title compound.

Yield: 0.18 g.

Mass spectrum (m/z, +ve ion mode): 448 (M⁺+1).

The following compounds were prepared similarly by coupling appropriate acid (racemic or pure enantiomer) with an appropriate amine, as applicable in each case:
(3-Benzyl-3-azabicyclo[3.2.1]oct-8-yl)methyl hydroxy(4-methylphenyl)phenylacetate (Compound No. 16),

Mass spectrum (m/z, +ve ion mode): 455.9 (M⁺+1);
(3-Benzyl-3-azabicyclo[3.2.1]oct-8-yl)methyl cycloheptyl(hydroxy)-2-thienylacetate (Compound No. 25),

Mass spectrum (m/z, +ve ion mode): 462 (M⁺+1);
(3-Benzyl-3-azabicyclo[3.2.1]oct-8-yl)methyl cyclohexyl(hydroxy)-2-thienylacetate (Compound No. 26),

Mass spectrum (m/z, +ve ion mode): 454 (M⁺+1);
(3-Benzyl-3-azabicyclo[3.2.1]oct-8-yl)methyl cycloheptyl(hydroxy)phenylacetate (Compound No. 27),

Mass spectrum (m/z, +ve ion mode): 462 (M⁺+1);
(3-Benzyl-3-azabicyclo[3.2.1]oct-8-yl)methyl (4-fluorophenyl)(hydroxy)phenylacetate (Compound No. 28),

Mass spectrum (m/z, +ve ion mode): 460 (M⁺+1);
(3-Benzyl-3-azabicyclo[3.2.1]oct-8-yl)methyl (2R)-[(1R)-3,3-difluorocyclohexyl](hydroxy)phenylacetate (Compound No. 43).

### Example 1a

### Synthesis of (2R)—N-[(3-Benzyl-3-azabicyclo[3.2.1]oct-8-yl)methyl]-2-cyclopentyl-2-hydroxy-N-methyl-2-phenylacetamide (Compound No 14)

Hydroxybenzotriazole (0.304 g, 2.25 mmol), N-methylmorpholine (0.45 ml, 4.09 mmol) and 1-(3-dimethylaminopropyl)-3-ethyl carbodiimide hydrochloride (0.392 g, 2.049 mmol) was added to a solution of (2R)-cyclopentyl(hydroxy)phenylacetic acid (0.45 g, 2.049 mmol) and 1-(3-benzyl-3-azabicyclo[3.2.1]oct-8-yl)-N-methylmethanamine (0.50 g, 2.049 mmol) in chloroform. The reaction mixture was stirred overnight at room temperature. The reaction mixture was poured into water and the resulting organic layer was separated. The organic layer was dried over anhydrous sodium sulfate, concentrated under reduced pressure and the residue thus obtained was purified by column chromatography to yield the title compound.

Mass spectrum (m/z, +ve ion mode): 447 (M⁺+1).

The following compound was prepared similarly using the appropriate corresponding reagents:
3-Hydroxy-N-{[3-(4-methylbenzyl)-3-azabicyclo[3.2.1]oct-8-yl]methyl}-2-phenylpropanamide (Compound No. 24),

Mass spectrum (m/z, +ve ion mode): 393 (M⁺+1).

### Scheme II

### Example 2

### Synthesis of (3-methyl-3-azabicyclo[3.2.1]oct-8-yl)methyl hydroxy(4-methylphenyl)phenylacetate (Compound No. 29)

### Step a: Synthesis of 3-azabicyclo[3.2.1]oct-8-ylmethyl hydroxy(4-methylphenyl)phenylacetate

Ammonium formate (112 mg) and palladium on carbon (10%) was added to a solution of Compound No. 16 (160 mg) in methanol (15 ml) and the reaction mixture was refluxed for 60 minutes. The reaction mixture was filtered over a celite pad. The resulting filtrate was concentrated under reduced pressure and the residue thus obtained was basified with aqueous sodium hydroxide solution and extracted with ethyl acetate. The resulting organic layer was washed with brine, dried over anhydrous sodium sulfate and concentrated under reduced pressure to yield the title compound.

### Step b: Synthesis of (3-methyl-3-azabicyclo[3.2.1]oct-8-yl)methyl hydroxy(4-methylphenyl)phenylacetate (Compound No. 29)

Formaldehyde (1.5 ml) and sodium cyanoborohydride (78 mg) was added to solution of the compound obtained from step a (90 mg) above in acetonitrile (15 ml) and the reaction mixture was stirred overnight at room temperature. The reaction mixtures was neutralized with acetic acid and stirred at room temperature for 30 minutes. The reaction mixture was concentrated under reduced pressure and a residue thus obtained was purified by column chromatography using 3% methanol in dichloromethane and 1% ammonia solvent mixture as eluent to yield the title compound. Yield: 50 mg.

Mass spectrum (m/z, +ve ion mode): 455.9 (M⁺+1).

The following compounds were prepared similarly using the appropriate corresponding reagents:
(3-Methyl-3-azabicyclo[3.2.1]oct-8-yl)methyl cycloheptyl(hydroxy)phenylacetate (Compound No. 30);
(3-Methyl-3-azabicyclo[3.2.1]oct-8-yl)methyl (4-fluorophenyl)(hydroxy)phenylacetate (Compound No. 31),

Mass spectrum (m/z, +ve ion mode): 384.2 (M⁺+1);
(3-Methyl-3-azabicyclo[3.2.1]oct-8-yl)methyl (2R)-[(1R)-3,3-difluorocyclopentyl](hydroxy)phenylacetate (Compound No. 32),

Mass spectrum (m/z, +ve ion mode): 394.19 M⁺+1);
(3-Methyl-3-azabicyclo[3.2.1]oct-8-yl)methyl cyclohexyl(hydroxy)phenylacetate (Compound No. 33),

Mass spectrum (m/z, +ve ion mode): 372 (M⁺+1);
(3-Methyl-3-azabicyclo[3.2.1]oct-8-yl)methyl (2R)-[(1S)-3,3-difluorocyclopentyl](hydroxy)phenylacetate (Compound No. 44);
(3-Methyl-3-azabicyclo[3.2.1]oct-8-yl)methyl (2R)-[(1R)-3,3-difluorocyclohexyl](hydroxy)phenylacetate (Compound No. 45);
(3-Methyl-3-azabicyclo[3.1.0]hex-6-yl)methyl (2R)-[(1S)-3,3-difluorocyclohexyl](hydroxy)phenylacetate (Compound No. 46),

Mass spectrum (m/z, +ve ion mode): 408 (M⁺+1);
(3-Methyl-3-azabicyclo[3.1.0]hex-6-yl)methyl (2R)-(3,3-difluorocyclopentyl)(hydroxy)phenylacetate (Compound No. 47),

Mass spectrum (m/z, +ve ion mode): 366 (M⁺+1).

### Scheme III

### Example 3

### Synthesis of iodide salt of 3-benzyl-6-({[cyclopentyl(hydroxy)-2-thienylacetyl]amino}methyl)-3-methyl-3-azoniabicyclo[3.1.0]hexane (Compound No. 1)

Methyl iodide (excess) was added to a solution of N-[(3-benzyl-3-azabicyclo[3.1.0]hex-6-yl)methyl]-2-cyclopentyl-2-hydroxy-2-(2-thienyl)acetamide (disclosed in WO 2006/117754) (50 mg) in dichloromethane (0.5 ml) and the reaction mixture was stirred overnight at room temperature. The reaction mixture was concentrated under reduced pressure and diethyl ether was added to the concentrate. The precipitate thus formed was macerated with diethyl ether to yield the title compound.

Yield: 45 mg.

Mass spectrum (m/z, +ve ion mode): 425 (M⁺+1).

The following compounds were prepared similarly using the appropriate corresponding reagents:
Iodide salt of 3-cyclohexylmethyl-6-({[cyclopentyl(hydroxy)phenylacetyl]amino}methyl)-3-methyl-3-azoniabicyclo[3.1.0]hexane (Compound No. 2),

Mass spectrum (m/z, +ve ion mode): 425.16 (M⁺);
Iodide salt of 6-({[hydroxy(3-methylphenyl)phenylacetyl]oxy}methyl)-3,3-dimethyl-3-azoniabicyclo[3.1.0]hexane (Compound No. 3),

Mass spectrum (m/z, +ve ion mode): 366 (M⁺);
Iodide salt of 3-benzyl-6-({[cyclopentyl(hydroxy)(4-methylphenyl)acetyl]amino}methyl)-3-methyl-3-azoniabicyclo[3.1.0]hexane (Compound No. 4),

Mass spectrum (m/z, +ve ion mode): 433.15 (M⁺);
Iodide salt of 6-({[hydroxy(diphenyl)acetyl]amino}methyl)-3-methyl-3-(4-methylbenzyl)-3-azoniabicyclo[3.1.0]hexane (Compound No. 5),

Mass spectrum (m/z, +ve ion mode): 441.15 (M⁺);
Iodide salt of 6-({[cyclopentyl(hydroxy)phenylacetyl]amino}methyl)-3-methyl-3-(3-methylbenzyl)-3-azoniabicyclo[3.1.0]hexane (Compound No. 6),

Mass spectrum (m/z, +ve ion mode): 433.22 (M⁺);
Iodide salt of 3-benzyl-6-({[hydroxy(4-methylphenyl)phenylacetyl]oxy}methyl)-3-methyl-3-azoniabicyclo[3.1.0]hexane (Compound No. 7),

Mass spectrum (m/z, +ve ion mode): 442.15 (M⁺);
Iodide salt of 3-benzyl-6-({[cyclopentyl(hydroxy)-2-thienylacetyl]oxy}methyl)-3-methyl-3-azoniabicyclo[3.1.0]hexane (Compound No. 8),

Mass spectrum (m/z, +ve ion mode): 426 (M⁺);
Iodide salt of 3-benzyl-6-({[hydroxy(2-dithienyl)acetyl]amino}methyl)-3-methyl-3-azoniabicyclo[3.1.0]hexane (Compound No. 9),

Mass spectrum (m/z, +ve ion mode): 440 (M⁺);
Iodide salt of 3-benzyl-8-({[cyclohexyl(hydroxy)phenylacetyl]oxy}methyl)-3-methyl-3-azoniabicyclo[3.2.1]octane (Compound No. 10),

Mass spectrum (m/z, +ve ion mode): 462 (M⁺);
Bromide salt of 3-benzyl-6-({[hydroxy(phenyl)-2-thienylacetyl]oxy}methyl)-3-methyl-3-azoniabicyclo[3.1.0]hexane (Compound No. 12),

Mass spectrum (m/z, +ve ion mode): 434 (M⁺);
Iodide salt of 3-(3-fluorobenzyl)-6-({[hydroxy(diphenyl)acetyl]amino}methyl)-3-methyl-3-azoniabicyclo[3.1.0]hexane (Compound No. 13),

Mass spectrum (m/z, +ve ion mode): 445.14 (M⁺);
Iodide salt of 3-benzyl-8-{[[(2R)-2-cyclopentyl-2-hydroxy-2-phenylacetyl](methyl)amino]methyl}-3-methyl-3-azoniabicyclo[3.2.1]octane (Compound No. 15),

Mass spectrum (m/z, +ve ion mode): 461 (M⁺);
Iodide salt of 3-benzyl-8-({[hydroxy(4-methylphenyl)phenylacetyl]oxy}methyl)-3-methyl-3-azoniabicyclo[3.2.1]octane (Compound No. 17),

Mass spectrum (m/z, +ve ion mode): 470 (M⁺);
Iodide salt of 3-benzyl-6-({[(4-fluorophenyl)(hydroxy)phenylacetyl]amino}methyl)-3-methyl-3-azoniabicyclo[3.1.0]hexane (Compound No. 18),

Mass spectrum (m/z, +ve ion mode): 445.18 (M⁺);
Iodide salt of 3-benzyl-6-({[(4-fluorophenyl)(hydroxy)phenylacetyl]oxy}methyl)-3-methyl-3-azoniabicyclo[3.1.0]hexane (Compound No. 19),

Mass spectrum (m/z, +ve ion mode): 446.12 (M⁺);
Iodide salt of 6-({[cyclopentyl(hydroxy)phenylacetyl]amino}methyl)-3-methyl-3-(4-methylbenzyl)-3-azoniabicyclo[3.1.0]hexane (Compound No. 20),

Mass spectrum (m/z, +ve ion mode): 433.29 (M⁺);
Iodide salt of 6-[({(2R)-2-[(1S)-3,3-difluorocyclohexyl]-2-hydroxy-2-phenylacetyl}oxy)methyl]-3,3-dimethyl-3-azoniabicyclo[3.1.0]hexane (Compound No. 21),

Mass spectrum (m/z, +ve ion mode): 394 (M⁺+1);
Iodide salt of 6-({[(2R)-2-(3,3-difluorocyclopentyl)-2-hydroxy-2-phenylacetyl]oxy}methyl)-3,3-dimethyl-3-azoniabicyclo[3.1.0]hexane (Compound No. 22),

Mass spectrum (m/z, +ve ion mode): 380 (M⁺+1);
Iodide salt of 8-{[(3-hydroxy-2-phenylpropanoyl)amino]methyl}-3-methyl-3-(4-methylbenzyl)-3-azoniabicyclo[3.2.1]octane (Compound No. 23),

Mass spectrum (m/z, +ve ion mode): 407 (M⁺);
Iodide salt of 3-benzyl-8-({[2-cycloheptyl-2-hydroxy-2-(2-thienyl)acetyl]oxy}methyl)-3-methyl-3-azoniabicyclo[3.2.1]octane (Compound No. 34),

Mass spectrum (m/z, +ve ion mode): 482 (M);
Iodide salt of 3-benzyl-8-({[2-cyclohexyl-2-hydroxy-2-(2-thienyl)acetyl]oxy}methyl)-3-methyl-3-azoniabicyclo[3.2.1]octane (Compound No. 35),

Mass spectrum (m/z, +ve ion mode): 468 (M⁺);
Iodide salt of 3-benzyl-8-({[2-cycloheptyl-2-hydroxy-2-phenylacetyl]oxy}methyl)-3-methyl-3-azoniabicyclo[3.2.1]octane (Compound No. 36),

Mass spectrum (m/z, +ve ion mode): 476 (M⁺);
Iodide salt of 3-benzyl-8-({[(4-fluorophenyl)(hydroxy)phenylacetyl]oxy}methyl)-3-methyl-3-azoniabicyclo[3.2.1]octane (Compound No. 37),

Mass spectrum (m/z, +ve ion mode): 474 (M⁺);
Iodide salt of 8-({[hydroxy(4-methylphenyl)phenylacetyl]oxy}methyl)-3,3-dimethyl-3-azoniabicyclo[3.2.1]octane (Compound No. 38),

Mass spectrum (m/z, +ve ion mode): 394.2 (M⁺);
Iodide salt of 8-({[cycloheptyl(hydroxy)phenylacetyl]oxy}methyl)-3,3-dimethyl-3-azoniabicyclo[3.2.1]octane (Compound No. 39),

Mass spectrum (m/z, +ve ion mode): 400 (M⁺);
Iodide salt of 8-({[(4-fluorophenyl)(hydroxy)phenylacetyl]oxy}methyl)-3,3-dimethyl-3-azoniabicyclo[3.2.1]octane (Compound No. 40),

Mass spectrum (m/z, +ve ion mode): 398 (M⁺+1);
Iodide salt of 8-[({(2R)-2-[(1R)-3,3-difluorocyclopentyl]-2-hydroxy-2-phenylacetyl}oxy)methyl]-3,3-dimethyl-3-azoniabicyclo[3.2.1]octane (Compound No. 41),

Mass spectrum (m/z, +ve ion mode): 408 (M⁺+1);
Iodide salt of 8-({[cyclohexyl(hydroxy)phenylacetyl]oxy}methyl)-3,3-dimethyl-3-azoniabicyclo[3.2.1]octane (Compound No. 42),

Mass spectrum (m/z, +ve ion mode): 386 (M⁺+1);
Iodide salt of 8-[({(2R)-2-[(1S)-3,3-difluorocyclopentyl]-2-hydroxy-2-phenylacetyl}oxy)methyl]-3,3-dimethyl-3-azoniabicyclo[3.2.1]octane (Compound No. 48),

Mass spectrum (m/z, +ve ion mode): 408 (M⁺+1);
Iodide salt of 8-[({(2R)-2-[(1R)-3,3-difluorocyclohexyl]-2-hydroxy-2-phenylacetyl}oxy)methyl]-3,3-dimethyl-3-azoniabicyclo[3.2.1]octane (Compound No. 49),

Mass spectrum (m/z, +ve ion mode): 422 (M⁺+1);
Iodide salt of 3-benzyl-8-[({(2R)-2-[(1R)-3,3-difluorocyclohexyl]-2-hydroxy-2-phenylacetyl}oxy)methyl]-3-methyl-3-azoniabicyclo[3.2.1]octane (Compound No. 50),

Mass spectrum (m/z, +ve ion mode): 498 (M⁺+1).

### Biological Activity

### In-Vitro Experiments

### Radioligand Binding Assays:

The affinity of test compounds for M₂ and M₃ muscarinic receptor subtypes was determined by [³H]-N-Methylscopolamine (NMS) binding studies using rat heart and submandibular gland respectively as described by Moriya et al., Life Sci, 1999, 64(25): 2351-2358 with minor modifications. Specific binding of [³H]-NMS was also determined using membranes from Chinese hamster ovary (CHO) cells expressing cloned human muscarinic receptor subtypes.

### Membrane Preparation:

### (a) Rat Tissues

Submandibular glands and heart were isolated and placed in ice-cold homogenizing buffer (HEPES 20 mM, 10 mM EDTA, pH 7.4) immediately after sacrifice. The tissues were homogenized in ten volumes of homogenizing buffer and the homogenate was filtered through two layers of wet gauze and filtrate was centrifuged at 500 g for 10 minutes. The supernatant was subsequently centrifuged at 40,000 g for 20 minutes. The pellet thus obtained was resuspended in assay buffer (HEPES 20 mM, EDTA 5 mM, pH 7.4) and were stored at −70° C. until the time of assay.

### (b) CHO Cells Expressing Human Recombinant Receptors

The cell pellets were homogenized for 30 seconds at 12,000 to 14,000 rpm, with intermittent gaps of 10-15 seconds in ice-cold homogenizing buffer (20 mM HEPES, 10 mM EDTA, pH 7.4). The homogenate was then centrifuged at 40,000 g for 20 minutes at 4° C. The pellet thus obtained was resuspended in homogenizing buffer containing 10% sucrose and was stored at −70° C. until the time of assay.

### Ligand Binding Assay:

The test compounds were dissolved and diluted in dimethyl sulfoxide. The membrane homogenates (5-10 μg protein) were incubated in 250 μL of assay buffer (20 mM HEPES, pH 7.4) at 24-25° C. for 3 hrs. Non-specific binding was determined in the presence of 1 μM Atropine. The incubation was terminated by vacuum filtration over GF/B fiber filter mats (Wallac) using Skatron cell harvester. The filters were then washed with ice-cold 50 mM Tris HCl buffer (pH 7.4). The filter mats were dried and transferred to 24 well plates (PET A No Cross Talk) followed by addition of 500 μl of scintillation cocktail. Radioactivity retained on filters was counted in Microbeta scintillation counter. The IC₅₀ & Kd were estimated by using the non-linear curve-fitting program using GraphPad Prism software. The value of inhibition constant, Ki was calculated from competitive binding studies by using Cheng & Prusoff's equation (Biochem Pharmacol, 22:3099-3108 (1973)), Ki=IC₅₀/(1+[L]/Kd), wherein [L] is the concentration of ligand [³H]-N-methyl scopolamine used in the particular experiment and Kd is the estimate of affinity of receptors to the ligand.

Tested compounds exhibited Ki values for M₃ and M₂ receptors of from about 20 nM to about 4000 nM; from about 20 nM to about 500 nM; from about 20 nM to about 200 nM; and from about 20 nM to about 50 nM.

### Functional Experiments Using Isolated Rat Bladder:

### Methodology:

Animals are euthanized by overdose of thiopentone and whole bladder is isolated and removed rapidly and placed in ice cold Tyrode buffer with the following composition (mmol L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder is cut into longitudinal strips (3 mm wide and 5-6 mm long) and mounted in 10 ml organ baths at 30° C., with one end connected to the base of the tissue holder and the other end connected through a force displacement transducer. Each tissue is maintained at a constant basal tension of 1 g and allowed to equilibrate for 1.5 hours during which the Tyrode buffer is changed every 15-20 minutes. At the end of equilibration period, the stabilization of the tissue contractile response is assessed with 1 μmol/L of carbachol until a reproducible response is obtained. Subsequently a cumulative concentration response curve to carbachol (10⁻⁹ mol/L to 3×10⁻⁴ mol/L) is obtained. After several washes and once a baseline is achieved, a cumulative concentration response curve is obtained in presence of NCE (NCE added 20 minutes prior to the second cumulative response curve.

The contractile results are expressed as % of control Emax. ED50 values are calculated by fitting a non-linear regression curve (Graph Pad Prism). pKb values are calculated by the formula pKb=−log [(molar concentration of antagonist/(dose ratio−1))] where, dose ratio=ED50 in the presence of antagonist/ED50 in the absence of antagonist.

### In-Vitro Functional Assay to Evaluate Efficacy of MRA on Guinea Pig & Rat Trachea

### Animals and Anaesthesia

Trachea tissue is obtained from guinea pigs (under an overdose of anesthesia (sodium pentobarbital, ˜300 mg/kg i.p) and immediately kept in an ice-cold Krebs Henseleit buffer of the following composition (mM): NaCl, 118; KCl 4.7; CaCl₂, 2.5; MgSO₄, 1.2; NaHCO₃, 25; KH₂PO₄, 1.2, glucose 11.1.

### Trachea Experiments:

Trachea tissue is cleaned off adherent fascia and cut into seven to eight strips of equal size (with approximately 4-5 tracheal rings in each strip). The trachea is opened along the mid-dorsal surface with the smooth muscle band intact and a series of transverse cuts from alternate sides is made so that they did not transect the preparation completely. The opposite end of the cut rings are tied using thread. The tissue is mounted in isolated tissue baths containing 10 mL Krebs Henseleit buffer maintained at 37° C. and bubbled with carbogen (95% oxygen and 5% carbon dioxide), at a basal tension of 1 gm. The buffer is changed 3-4 times for about an hour. The tissues are equilibrated for 1 hour for stabilization. After 1 hour, the tissue is contacted with 60 mM KCl. This procedure is repeated after every 2-3 washes until two similar consecutive responses are obtained. At the end of stabilization, a carbachol concentration-response curve is performed on all the tissues. The tissues were washed until the baseline is obtained. Thereafter, each tissue was incubated with different concentrations of MRA/Standard/Vehicle for 20 minutes followed by a second cumulative dose response curve to carbachol. The contractile response of tissues is recorded either on a Powerlab system or on Grass polygraph (Model 7). The responses to carbachol were standardized as a percentage of the maximum carbachol response of the control CRC. The carbachol EC₅₀ values in the presence and absence of inhibitor are determined using graph pad prism. pK_{B} values, an index of functional antagonism from EC₅₀ data, were calculated using the following relationship:

−log [antagonist (M)/(EC₅₀ antagonist/EC₅₀ control)−1]

The data is expressed as mean±s.e.m for n observations. In tissues where Eₘₐₓ attained is less than 50%, pK_{B} is calculated by Kenakin's double reciprocal plot.

All drugs and chemicals used in the study are of AR grade. Carbachol is procured from Sigma Chemicals, U.S.A. Stock solutions of Standard/New Chemical Entities (NCEs) are prepared in DMSO. Subsequent dilutions are prepared from the stock in MilliQ water.

In-Vitro Functional Assay to Evaluate Efficacy of “MRA” in Combination with “PDE-IV Inhibitors”

### Animals and Anaesthesia:

Trachea tissue is obtained from a guinea pig (400-600 gm) under anesthesia (sodium pentobarbital, 300 mg/kg i.p) and is immediately kept in an ice-cold Krebs Henseleit buffer. Indomethacin (10 uM) is present throughout the KH buffer to prevent the formation of bronchoactive prostanoids.

### Trachea Experiments:

Trachea tissue is cleaned off adherent fascia and cut it into strips of equal size (with approx. 4-5 tracheal rings in each strip). The epithelium is removed by careful rubbing, minimizing damage to the smooth muscle. The trachea is opened along the mid-dorsal surface with the smooth muscle band intact and a series of transverse cuts is made from alternate sides so that they do not transect the preparation completely. Opposite ends of the cut rings are tied with the help of a thread. The tissue is mounted in isolated tissue baths containing 10 mL Krebs Henseleit buffer maintained at 37° C. and is bubbled with carbogen, at a basal tension of 1 gm. The buffer is changed 4-5 times for about an hour and the tissue is equilibrated for 1 hour for stabilization. After 1 hour, the tissue is contacted with 1 uM carbachol. Repeat this after every 2-3 washes until two similar consecutive responses are obtained. At the end of stabilization, the tissue is washed for 30 minutes followed by incubation with suboptimal dose of MRA/Vehicle for 20 minutes prior to contraction of the tissues with 1 μM carbachol. The relaxant activity of the PDE-IV inhibitor [10⁻⁹M to 10⁻⁴M] on the stabilized developed tension/response is assessed. The contractile response of tissues is recorded either on a Powerlab data acquisition system or on a Grass polygraph (Model 7). The relaxation is expressed as a percentage of maximum carbachol response. The data is expressed as mean±s.e. mean for n observations. The EC₅₀ is calculated as the concentration producing 50% of the maximum relaxation to 1 μM carbachol. The percent relaxation between the treated and control tissues is compared using non-parametric unpaired t-test. A p value of <0.05 is considered to be statistically significant.

### In-Vivo Experiments

### In-Vivo Assay to Evaluate Efficacy of MRA Inhibitors

Male guinea pigs are anesthetized with urethane (1.5 g/kg, i.p.). Trachea is cannulated along with jugular vein (for carbachol challenge) and animals are placed in a Plethysmograph-Box (PLY 3114 model; Buxco Electronics, Sharon, USA.). Respiratory parameters are recorded using Pulmonary Mechanics Analyser, Biosystems XA software (Buxco Electronics, USA), which calculates lung resistance (R_{L}) on a breath-by-breath basis. Bronchoconstriction is induced by injections of Carbachol (10 μg/kg) delivered into the jugular vein. Increase in R_{L} over a period of 5 minutes post carbachol challenge is recorded in presence or absence of MRA or vehicle at 2 hours and 12 hours post treatment and expressed as % increase in R_{L} from basal.

$$\% Inhibition = \frac{{R}_{L vehicle} - {R}_{L test}}{{R}_{L vehicle}} \times 100$$

### Where

R_{L} vehicle % increase in lung resistance from basal in vehicle treated
R_{L} test % increase in lung resistance from basal at a given dose of test
   In-Vivo Assay to Evaluate Efficacy of MRA in Combination with PDE-IV Inhibitors

### Drug Treatment:

MRA (1 μg/kg to 1 mg/kg) and PDE-IV inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 g/mL) for 40 minutes. One group of vehicle treated rats is exposed to phosphate buffered saline (PBS) for 40 minutes. Two hours after LPS/PBS exposure, animals are placed inside a whole body plethysmograph (Buxco Electronics, USA) and exposed to PBS or increasing acetylcholine (1, 6, 12, 24, 48 and 96 mg/mL) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyper-reactivity response is recorded, animals are sacrificed and bronchoalveolar lavage (BAL) is performed. Collected lavage fluid is centrifuged at 3000 rpm for 5 minutes, at 4° C. The resulting pellet is collected and resuspended in 1 mL HBSS. Total leukocyte count is performed in the resuspended sample. A portion of suspension is cytocentrifuged and stained with Leishmann's stain for differential leukocyte count. Total leukocyte and neutrophil counts are expressed as cell count (millions cells mL⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
The percent inhibition data is used to compute ED₅₀ vales using Graph Pad Prism software (Graphpad Software Inc., USA).
   In-Vivo Assay to Evaluate Efficacy of MRA in Combination with Corticosteroids Ovalbumin Induced Airway Inflammation:

Guinea pigs are sensitized on days 0, 7 and 14 with 50-μg ovalbumin and 10 mg aluminum hydroxide is injected intraperitoneally. On days 19 and 20 guinea pigs are exposed to 0.1% w v⁻¹ ovalbumin or PBS for 10 minutes, and with 1% ovalbumin for 30 minutes on day 21. Guinea pigs are treated with test compound (0.1, 0.3 and 1 mg kg-1) or standard 1 mg kg⁻¹ or vehicle once daily from day 19 and continued for 4 days. Ovalbumin/PBS challenge is performed 2 hours after different drug treatment.

24 hrs after the final ovalbumin challenge, BAL is performed using Hank's balanced salt solution (HBSS). Collected lavage fluid is centrifuged at 3000 rpm for 5 minutes, at 4° C. The resulting pellet is collected and resuspended in 1 mL HBSS. Total leukocyte count is performed in the resuspended sample. A portion of suspension is cytocentrifuged and stained with Leishmann's stain for differential leukocyte count. Total leukocyte and eosinophil count are expressed as cell count (millions cells mL⁻¹ of BAL). Eosinophil is also expressed as percent of total leukocyte count. % inhibition is computed using the following formula.

$$\% Inhibition = \frac{{Eos}_{OVA} - {Eos}_{TEST}}{{Eos}_{OVA} - {Eos}_{CON}} \times 100$$

### Where,

Eos_{OVA}=Percentage of eosinophil in untreated ovalbumin challenged group
Eos_{TEST}=Percentage of eosinophil in group treated with a given dose of test compound
Eos_{CON}=Percentage of eosinophil in group not challenged with ovalbumin.
   In-Vivo Assay to Evaluate Efficacy of “MRA” in Combination with p38 Map Kinase Inhibitors

Lipopolysaccharide (LPS) induced airway hyper reactivity (AHR) and neutrophilia:

### Drug Treatment:

MRA (1 μg/kg to 1 mg/kg) and p38 MAP kinase inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/mL) for 40 minutes. One group of vehicle treated rats is exposed to phosphate buffered saline (PBS) for 40 minutes. Two hours after LPS/PBS exposure, animals are placed inside a whole body plethysmograph (Buxco Electronics, USA) and exposed to PBS or increasing acetylcholine (1, 6, 12, 24, 48 and 96 mg/mL) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyper reactivity response is recorded, animals are sacrificed and bronchoalveolar lavage (BAL) is performed. Collected lavage fluid is centrifuged at 3000 rpm for 5 minutes, at 4° C. The resulting pellet is collected and resuspended in 1 mL HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells mL¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
   The percent inhibition data is used to compute ED₅₀ vales using Graph Pad Prism software (Graphpad Software Inc., USA).
   In-Vivo Assay to Evaluate Efficacy of “MRA” in Combination with β2-Agonists

### Drug Treatment:

MRA (1 μg/kg to 1 mg/kg) and long acting P2 agonist are instilled intratracheally under anesthesia either alone or in combination.

### Method

Wistar rats (250-350 gm) or balb/C mice (20-30 gm) are placed in body box of a whole body plethysmograph (Buxco Electronics., USA) to induce bronchoconstriction. Animals are allowed to acclimatize in the body box and are given successive challenges, each of 2 minutes duration, with PBS (vehicle for acetylcholine) or acetylcholine (i.e. 24, 48, 96, 144, 384, and 768 mg/mL). The respiratory parameters are recorded online using Biosystem XA software, (Buxco Electronics, USA) for 3 minutes. A gap of 2 minutes is allowed for the animals to recover and then challenged with the next higher dose of acetylcholine (ACh). This step is repeated until Penh of rats attained 2 times the value (PC-100) seen with PBS challenge. Following PBS/ACh challenge, Penh values (index of airway resistance) in each rat/mice is obtained in the presence of PBS and different doses of ACh. Penh, at any chosen dose of ACh is, expressed as percent of PBS response. The Penh values thus calculated are fed into Graph Pad Prism software (Graphpad Software Inc., USA) and using a nonlinear regression analysis PC100 (2 folds of PBS value) values are computed. % inhibition is computed using the following formula.

$$\% Inhibition = \frac{PC {100}_{TEST} - PC {100}_{CON}}{768 - PC {100}_{CON}} \times 100$$

### Where,

PC100_{CON}=PC100 in vehicle treated group
PC100_{TEST}=PC100 in group treated with a given dose of test compound
768=is the maximum amount of acetylcholine used.

1. A compound having the structure of Formula I: 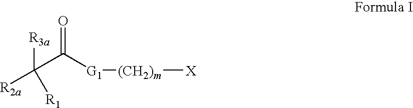
or a pharmaceutically accepted salt, pharmaceutically acceptable solvate, enantiomers, diastereomer, polymorph or N-oxide thereof, wherein
X is 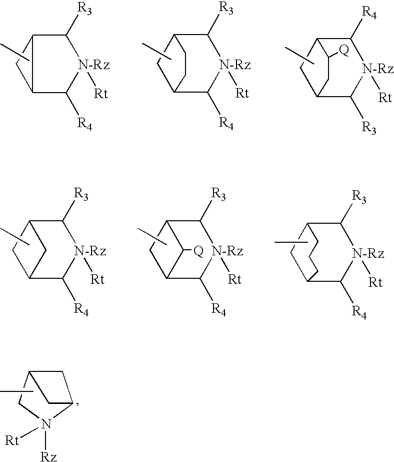
m is an integer selected from 0-4,
Q is alkyl, cycloalkyl, aryl, heteroaryl, heterocyclyl, aralkyl, heteroarylalkyl or heterocyclylalkyl,
G₁ is oxygen, sulfur, —NRᵤ, (wherein Rᵤ is hydrogen, alkyl, alkenyl, alkynyl, aryl, or aralkyl) or —CH₂—,
R₁ and R₂ₐ are independently selected from alkyl, alkenyl, alkynyl, cycloalkyl, aryl, aralkyl, heteroarylalkyl or heterocyclylalkyl,
R₃ and R₄ are independently selected from the group consisting of hydrogen, alkyl, alkenyl, alkynyl, cycloalkyl, aryl or aralkyl,
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy,
Rₓ, and R_{y}, are independently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring,
Rz is hydrogen or Rq,
Rq is alkyl, alkenyl, alkynyl, cycloalkyl, aryl, heteroaryl, heterocyclyl, heterocyclylalkyl, heteroarytalkyl or aralkyl,
Rt is no atom or Rq1, and
Rq1 is alkyl, aryl, cycloalkyl, heteroaryl, heterocyclyl, hoteroarylalkyl, heterocyclylalkyl or aralkyl,
wherein when Rz is Rq and Rt is Rq1, then the compound of Formula I is a quaternary ammonium salt.

2. The compound of claim 1, selected from:
Iodide salt of 3-benzyl-6-({[cyclopentyl(hydroxy)-2-thienylacetyl]amino}methyl)-3-methyl-3-azoniabicyclo[3.1.0]hexane (Compound No. 1),
Iodide salt of 3-cyclohexylmethyl-6-({[cyclopentyl(hydroxy)phenylacetyl]amino}methyl)-3-methyl-3-azoniabicyclo[3.1.0]hexane (Compound No. 2),
Iodide salt of 6-({[hydroxy(3-methylphenyl)phenylacetyl]oxy}methyl)-3,3-dimethyl-3-azoniabicyclo[3.1.0]hexane (Compound No. 3),
Iodide salt of 3-benzyl-6-({[cyclopentyl(hydroxy)(4-methylphenyl)acetyl]amino}methyl)-3-methyl-3-azoniabicyclo[3.1.0]hexane (Compound No. 4),
Iodide salt of 6-({[hydroxy(diphenyl)acetyl]amino}methyl)-3-methyl-3-(4-methylbenzyl)-3-azoniabicyclo[3.1.0]hexane (Compound No. 5),
Iodide salt of 6-({[cyclopentyl(hydroxy)phenylacetyl]amino}methyl)-3-methyl-3-(3-methylbenzyl)-3-azoniabicyclo[3.1.0]hexane (Compound No. 6),
Iodide salt of 3-benzyl-6-({[hydroxy(4-methylphenyl)phenylacetyl]oxy}methyl)-3-methyl-3-azoniabicyclo[3.1.0]hexane (Compound No. 7),
Iodide salt of 3-benzyl-6-({[cyclopentyl(hydroxy)-2-thienylacetyl]oxy}methyl)-3-methyl-3-azoniabicyclo[3.1.0]hexane (Compound No. 8),
Iodide salt of 3-benzyl-6-({[hydroxy(2-dithienyl)acetyl]amino}methyl)-3-methyl-3-azoniabicyclo[3.1.0]hexane (Compound No. 9),
Iodide salt of 3-benzyl-8-({[cyclohexyl(hydroxy)phenylacetyl]oxy}methyl)-3-methyl-3-azoniabicyclo[3.2.1]octane (Compound No. 10),
(3-Benzyl-3-azabicyclo[3.2.1]oct-8-yl)methyl cyclohexyl(hydroxy)phenylacetate (Compound No. 11),
Bromide salt of 3-benzyl-6-({[hydroxy(phenyl)-2-thienylacetyl]oxy}methyl)-3-methyl-3-azoniabicyclo[3.1.0]hexane (Compound No. 12),
Iodide salt of 3-(3-fluorobenzyl)-6-({[hydroxy(diphenyl)acetyl]amino}methyl)-3-methyl-3-azoniabicyclo[3.1.0]hexane (Compound No. 13),
(2R)—N-[(3-Benzyl-3-azabicyclo[3.2.1]oct-8-yl)methyl]-2-cyclopentyl-2-hydroxy-N-methyl-2-phenylacetamide (Compound No 14),
Iodide salt of 3-benzyl-8-{[[(2R)-2-cyclopentyl-2-hydroxy-2-phenylacetyl](methyl)amino]methyl}-3-methyl-3-azoniabicyclo[3.2.1]octane (Compound No. 15),
(3-Benzyl-3-azabicyclo[3.2.1]oct-8-yl)methyl hydroxy(4-methylphenyl)phenylacetate (Compound No. 16),
Iodide salt of 3-benzyl-8-({[hydroxy(4-methylphenyl)phenylacetyl]oxy}methyl)-3-methyl-3-azoniabicyclo[3.2.1]octane (Compound No. 17),
Iodide salt of 3-benzyl-6-({[(4-fluorophenyl)(hydroxy)phenylacetyl]amino}methyl)-3-methyl-3-azoniabicyclo[3.1.0]hexane (Compound No. 18),
Iodide salt of 3-benzyl-6-({[(4-fluorophenyl)(hydroxy)phenylacetyl]oxy}methyl)-3-methyl-3-azoniabicyclo[3.1.0]hexane (Compound No. 19),
Iodide salt of 6-({[cyclopentyl(hydroxy)phenylacetyl]amino}methyl)-3-methyl-3-(4-methylbenzyl)-3-azoniabicyclo[3.1.0]hexane (Compound No. 20),
Iodide salt of 6-[({(2R)-2-[(1S)-3,3-difluorocyclohexyl]-2-hydroxy-2-phenylacetyl}oxy)methyl]-3,3-dimethyl-3-azoniabicyclo[3.1.0]bexane (Compound No. 21),
Iodide salt of 6-({[(2R)-2-(3,3-difluorocyclopentyl)-2-hydroxy-2-phenylacetyl]oxy}methyl)-3,3-dimethyl-3-azoniabicyclo[3.1.0]hexane (Compound No. 22),
Iodide salt of 8-{[(3-hydroxy-2-phenylpropanoyl)amino]methyl}-3-methyl-3-(4-methylbenzyl)-3-azoniahicyclo[3.2.1]octane (Compound No. 23),
3-Hydroxy-N-{[3-(4-methylbenzyl)-3-azabicyclo[3.2.1]oct-8-yl]methyl}-2-phenylpropanamide (Compound No. 24),
(3-Benzyl-3-azabicyclo[3.2.1]oct-8-yl)methyl cycloheptyl(hydroxy)-2-thienylacetate (Compound No. 25),
(3-Benzyl-3-azabicyclo[3.2.1]oct-8-yl)methyl cyclohexyl(hydroxy)-2-thienylacetate (Compound No. 26),
(3-Benzyl-3-azabicyclo[3.2.1]oct-8-yl)methyl cycloheptyl(hydroxy)phenyl acetate (Compound No. 27),
(3-Benzyl-3-azabicyclo[3.2.1]oct-8-yl)methyl (4-fluorophenyl)(hydroxy)phenylacetate (Compound No. 28),
(3-Methyl-3-azabicyclo[3.2.1]oct-8-yl)methyl hydroxy(4-methylphenyl)phenylacetate (Compound No. 29),
(3-Methyl-3-azabicyclo[3.2.1]oct-8-yl)methyl cycloheptyl(hydroxy)phenylacetate (Compound No. 30), (3-Methyl-3-azabicyclo[3.2.1]oct-8-yl)methyl (4-fluorophenyl)(hydroxy)phenylacetate (Compound No. 31),
(3-Methyl-3-azabicyclo[3.2.1]oct-8-yl)methyl (2R)-[(1R)-3,3-difluorocyclopentyl](hydroxy)phenylacetate (Compound No. 32),
(3-Methyl-3-azabicyclo[3.2.1]oct-8-yl)methyl cyclohexyl(hydroxy)phenylacetate (Compound No. 33)),
Iodide salt of 3-benzyl-8-({[2-cycloheptyl-2-hydroxy-2-(2-thienyl)acetyl]oxy}methyl)-3-methyl-3-azoniabicyclo[3.2.1]octane (Compound No. 34),
Iodide salt of 3-benzyl-8-({[2-cyclohexyl-2-hydroxy-2-(2-thienyl)acetyl]oxy}methyl)-3-methyl-3-azoniabicyclo[3.2.1]octane (Compound No. 35),
Iodide salt of 3-benzyl-8-({[2-cycloheptyl-2-hydroxy-2-phenylacetyl]oxy}methyl)-3-methyl-3-azoniabicyclo[3.2.1]octane (Compound No. 36),
Iodide salt of 3-benzyl-8-({[(4-fluorophenyl)(hydroxy)phenylacetyl]oxy}methyl)-3-methyl-3-azoniabicyclo[3.2.1]octane (Compound No. 37),
Iodide salt of 8-({[hydroxy(4-methylphenyl)phenylacetyl]oxy}methyl)-3,3-dimethyl-3-azoniabicyclo[3.2.1]octane (Compound No. 38),
Iodide salt of 8-({[cycloheptyl(hydroxy)phenylacetyl]oxy}methyl)-3,3-dimethyl-3-azoniabicyclo[3.2.1]octane (Compound No. 39),
Iodide salt of 8-({[(4-fluorophenyl)(hydroxy)phenylacetyl]oxy}methyl)-3,3-dimethyl-3-azoniabicyclo[3.2.1]octane (Compound No. 40),
Iodide salt of 8-[({(2R)-2-[(1R)-3,3-difluorocyclopentyl]-2-hydroxy-2-phenylacetyl}oxy)methyl]-3,3-dimethyl-3-azoniabicyclo[3.2.1]octane (Compound No. 41),
Iodide salt of 8-({[cyclohexyl(hydroxy)phenylacetyl]oxy}methyl)-3,3-dimethyl-3-azoniabicyclo[3.2.1]octane (Compound No. 42),
(3-Benzyl-3-azabicyclo[3.2.1]oct-8-yl)methyl (2R)-[(1R)-3,3-difluorocyclohexyl](hydroxy)phenylacetate (Compound No. 43),
(3-Methyl-3-azabicyclo[3.2.1]oct-8-yl)methyl (2R)-[(1S)-3,3-difluorocyclopentyl](hydroxy)phenylacetate (Compound No. 44),
(3-Methyl-3-azabicyclo[3.2.1]oct-8-yl)methyl (2R)-[(1R)-3,3-difluorocyclohexyl](hydroxy)phenylacetate (Compound No. 45),
(3-Methyl-3-azabicyclo[3.1.0]hex-6-yl)methyl (2R)-[(1S)-3,3-difluorocyclohexyl](hydroxy)phenylacetate (Compound No. 46),
(3-Methyl-3-azabicyclo[3.1.0]hex-6-yl)methyl (2R)-(3,3-difluorocyclopentyl)(hydroxy)phenylacetate (Compound No. 47),
Iodide salt of 8-[({(2R)-2-[(1S)-3,3-difluorocyclopentyl]-2-hydroxy-2-phenylacetyl}oxy)methyl]-3,3-dimethyl-3-azoniabicyclo[3.2.1]octane (Compound No. 48),
Iodide salt of 8-[({(2R)-2-[(1R)-3,3-difluorocyclohexyl]-2-hydroxy-2-phenylacetyl}oxy)methyl]-3,3-dimethyl-3-azoniabicyclo[3.2.1]octane (Compound No. 49), and
Iodide salt of 3-benzyl-8-[({(2R)-2-[(1R)-3,3-difluorocyclohexyl]-2-hydroxy-2-phenylacetyl}oxy)methyl]-3-methyl-3-azoniabicyclo[3.2.1]octane (Compound No. 50).

3. A pharmaceutical composition comprising a therapeutically effective amount of a compound as defined in claim 1 and one or more pharmaceutically acceptable carriers, excipients or diluents.

4. A method of treating or preventing disease or disorder of the respiratory, urinary or gastrointestinal systems, wherein the disease or disorder is mediated through muscarinic receptors, comprising administering to a patient in need thereof a therapeutically effective amount of a compound of claim 1.

5. The method of claim 4, wherein the method is a method of treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

6. A pharmaceutical composition comprising one or more compounds of claim 1, 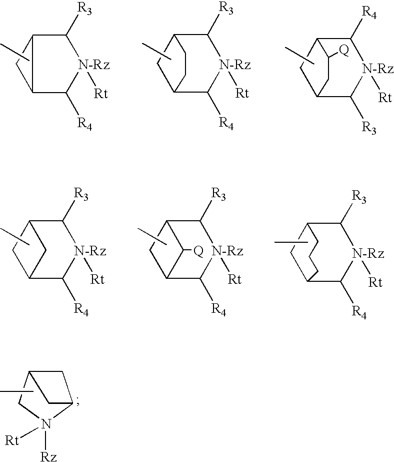
or a pharmaceutically accepted salt, pharmaceutically acceptable solvate, enantiomers, diastereomer, polymorph or N-oxide thereof further in combination with one or more corticosteroids, beta agonists, leukotriene antagonists, 5-lipoxygenase inhibitors, anti-histamines, antitussives, dopamine receptor antagonists, chemokine inhibitors, p38 MAP Kinase inhibitors, or PDE-IV inhibitors or a mixture thereof.

7. A method of preparing a compound of Formula V comprising the steps of:
a. reacting a compound of Formula II 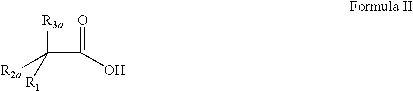
with a compound of Formula III 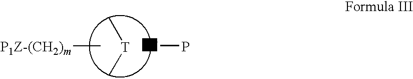
to form a compound of Formula IV, and 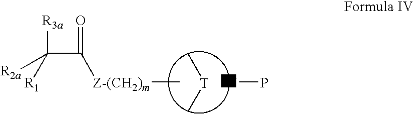
b. deprotecting a compound of Formula IV to form a compound of Formula V, 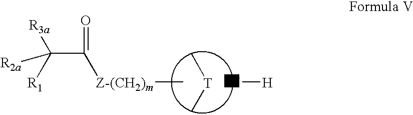
wherein
R₁ and R₂ₐ are independently selected from alkyl, alkenyl, alkynyl, cycloalkyl, aryl, aralkyl, heteroarylalkyl or heterocyclylalkyl;
R₃ₐ is hydrogen, hydroxyalkyl, —OSi(CH₃)₃₃ cyano, —CONRₓR_{y}, —COORₓ, hydroxy, alkoxy, alkenyloxy, alkynyloxy, aryloxy or cycloalkoxy;
Rₓ, and R_{y}, are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring;
P₁ is mesyl, tosyl or H when Z is oxygen or —NRᵤ, (wherein Rᵤ is hydrogen, alkyl, alkenyl, alkynyl, aryl, or aralkyl); or
P₁ is Br, Cl or I when Z is —CH₂;
m is an integer selected from 0-4;
P is a protecting group selected from aralkyl, —C(═O)Oaralkyl, —C(—O)OC(CH₃)₃, —C(═O)OC(CH₃)₂CHBr₂ or —C(═O)OC(CH₃)₂CCl₃; 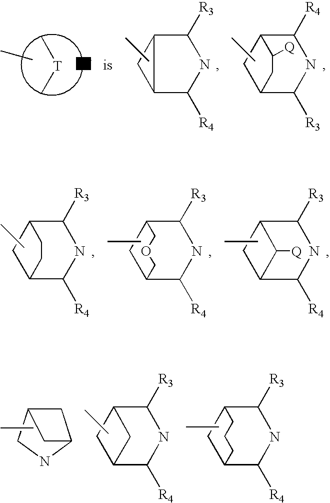
Q is alkyl, cycloalkyl, aryl, heteroaryl, heterocyclyl, aralkyl, heteroarylalkyl or heterocyclylalkyl; and
R₃ and R₄ are independently selected from the group consisting of hydrogen, alkyl, alkenyl, alkynyl, cycloalkyl, aryl or aralkyl.

8. A method of preparing a compound of Formula VII comprising the step of:
a. reacting a compound of Formula V 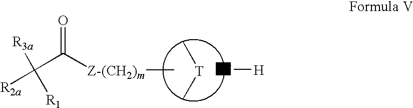
with a compound of Formula VI
Ra-L Formula VI
to form a compound of Formula VII, 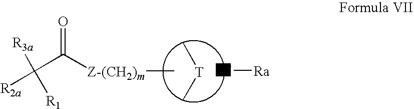
wherein
Z is oxygen or —NRᵤ (wherein Rᵤ is hydrogen, alkyl, alkenyl, alkynyl, aryl or aralkyl) or —CH₁;
R₁ and R₂ₐ are independently selected from alkyl, alkenyl, alkynyl, cycloalkyl, aryl, aralkyl, heteroarylalkyl or heterocyclylalkyl;
Ra is alkyl, alkenyl, alkynyl, heteroarylalkyl, heterocyclylalkyl, aralkyl or cycloalkyl;
L is a leaving group selected from halogen, triflate, tosylate or mesylate;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring;
m is an integer selected from 0-4; 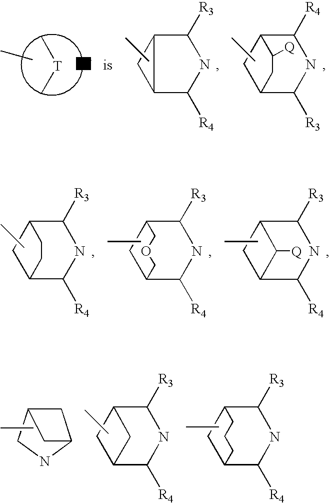
Q is alkyl, cycloalkyl, aryl, heteroaryl, heterocyclyl, aralkyl, heteroarylalkyl or heterocyclylalkyl;
R₃ and R₄ are independently selected from the group consisting of hydrogen, alkyl, alkenyl, alkynyl, cycloalkyl, aryl or aralkyl.

9. A method of preparing a compound of Formula VIII comprising the step of;
a. reacting a compound of Formula V 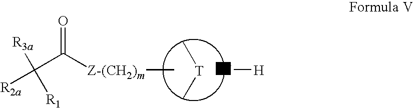
with a compound of Formula Va
Rb-CHO Formula Va
to form a compound of Formula VIII, 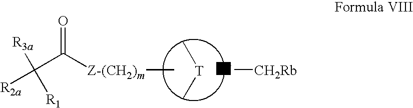
wherein
Z is oxygen or —NRᵤ, (wherein Rᵤ is hydrogen, alkyl, alkenyl, alkynyl, aryl, or aralkyl) or CH₂;
R₁ and R₂ₐ are independently selected from alkyl, alkenyl, alkynyl, cycloalkyl, aryl, aralkyl, heteroarylalkyl or heterocyclylalkyl;
Rb is hydrogen, alkyl, alkenyl, alkynyl, heteroarylalkyl, heterocyclylalkyl, aralkyl or cycloalkyl;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ, and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring;
m is an integer selected from 0-4; 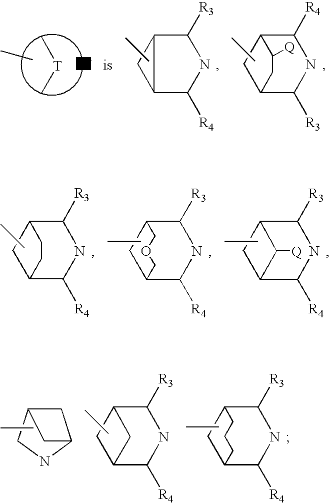
Q is alkyl, cycloalkyl, aryl, heteroaryl, heterocyclyl, aralkyl, heteroarylalkyl or heterocyclylalkyl; and
R₃ and R₄ are independently selected from the group consisting of hydrogen, alkyl, alkenyl, alkynyl, cycloalkyl, aryl or aralkyl.

10. A method of preparing a compound of Formula X comprising the step of:
a. reacting a compound of Formula IX 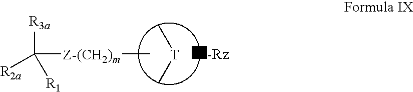
with a compound of Formula Rt-Rc to form a compound of Formula X, 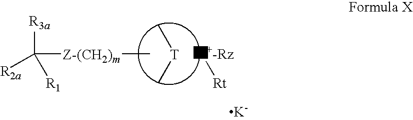
wherein
Z is oxygen or —NRᵤ (wherein Rᵤ is hydrogen, alkyl, alkenyl, alkynyl, aryl, or aralkyl) or —CH₂;
R₁ and R₂ₐ are independently selected from alkyl, alkenyl, alkynyl, cycloalkyl, aryl, aralkyl, heteroarylalkyl or heterocyclylalkyl;
Rt is no atom or Rq1;
Rq1 is alkyl, aryl, cycloalkyl, heteroaryl, heterocyclyl, heteroarylalkyl, heterocyclylalkyl or aralkyl;
Rc is halogen, mesyl, tosyl or triflyl;
R₃ₐ is hydrogen, hydroxyalkyl, —OSi(CH₃)₃, cyano, —CONRₓR_{y}, —COORₓ, hydroxy, alkoxy, alkenyloxy, alkynyloxy, aryloxy or cycloalkoxy;
Rₓ, and R_{y} are in dependently selected from hydrogen, alkyl, cycloalkyl, aryl, halogen, aralkyl, heteroaryl, heterocyclyl, heteroarylalkyl or heterocyclylalkyl; or Rₓ and R_{y} together join to form a heterocyclyl ring;
Rz is hydrogen or Rq;
Rq is alkyl, alkenyl, alkynyl, cycloalkyl, aryl, heteroaryl, heterocyclyl, heterocyclylalkyl, heteroarylalkyl or aralkyl;
K⁻ is an anion selected from tartrate, chloride, bromide, iodide, sulfate, phosphate, nitrate, carbonate, fumarate, glutamate, citrate, methanesulfonate, benzenesulfonate, maleate or succinate;
m is an integer selected from 0-4; 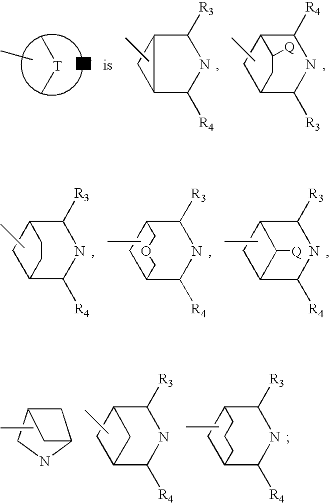
Q is alkyl cycloalkyl, aryl, heteroaryl, heterocyclyl, aralkyl, heteroarylalkyl or heterocyclylalkyl; and
R₃ and R₄ are independently selected from the group consisting of hydrogen, alkyl, alkenyl, alkynyl, cycloalkyl, aryl or aralkyl.
